(19) United States
(12) Patent Application Publication
(10) Pub. No.: US 2024/0018597 A1
(43) Pub. Date: Jan. 18, 2024

(54) DNA Copy Number Alterations (CNAs) to Determine Cancer Phenotypes

(71) Applicant: The University of North Carolina at Chapel Hill, Chapel Hill, NC (US)

(72) Inventors: Charles M. Perou, Chapel Hill, NC (US); Joel S. Parker, Apex, NC (US); Youli Xia, Chapel Hill, NC (US); Cheng Fan, Chapel Hill, NC (US)

(21) Appl. No.: 17/768,059

(22) PCT Filed: Oct. 9, 2020
(86) PCT No.: PCT/US20/55093

§ 371 Date: Apr. 11, 2022

Related U.S. Application Data
(60) Provisional application No. 62/912,727, filed on Oct. 9, 2019.

(51) Int. Cl. C12Q 1/6886; G16B 20/10; G16B 25/10; G16B 40/20; G16H 50/20; G16H 20/00

(52) U.S. Cl. CPC C12Q 1/6886; G16B 20/10; G16B 25/10; G16B 40/20; G16H 50/20; G16H 20/00; C12Q 2600/156; C12Q 2600/158; C12Q 2600/112

(57) ABSTRACT

The present disclosure provides a method for generating a calculated cancer signature for a cancer-related phenotype based on copy number alterations (CNAs) in a patient sample. The calculated cancer signature may correspond to a somatic mutation, an mRNA expression signature, or a protein expression signature. The disclosure also provides a method treating a patient using the calculated cancer phenotype. In addition, the disclosure provides a method for generating a calculated signature based on CNAs to replicate a cancer phenotype.

### CROSS REFERENCE TO RELATED APPLICATIONS

This application claims the benefit of U.S. Appn. No. 62/912,727 filed Oct. 9, 2019, Perou et al., Atty. Dkt. No. 150-32-PROV, which is hereby incorporated by reference in its entirety.

### STATEMENT REGARDING FEDERALLY SPONSORED RESEARCH OR DEVELOPMENT

This invention was made with government support under Grant Numbers CA58223, CA148761 and CA195740 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

This application contains a fifteen (15) tables and one (1) computer program listing as an appendix. They have been submitted electronically via EFS-Web as an ASCII text files. They have the following file attributes: (1) Supplementary Table 1, Annotation of Gene Expression Signatures entitled Supp_Table1.txt, it has a size of 2,042,486 bytes, and was created on Mar. 4, 2019; (2) Supplementary Table 2, Annotation of copy number segments entitled Supp_Table2.txt, it has a size of 728,822 bytes, and was created on Oct. 9, 2019; (3) Supplementary Table 3, Summary of Elastic Net models for gene entitled Supp_Table3.txt, it has a size of 1,236,182 bytes, and was created on Oct. 9, 2019; (4) Supplementary Table 4, Summary of Elastic Net models for molecular subtypes and histology in breast cancers entitled Supp_Table4.txt, it has a size of 37,355 bytes, and was created on Oct. 9, 2019; (5) Supplementary Table 5, Summary of Elastic Net models for protein expressions and clinical receptor statuses in breast cancers entitled Supp_Table5.txt, it has a size of 405,443 bytes, and was created on Oct. 9, 2019; (6) Supplementary Table 6, Summary of Elastic Net models for somatic mutations in breast cancers entitled Supp_Table6.txt, it has a size of 120,280 bytes, and was created on Oct. 9, 2019; (7) Supplementary Table 7, Summary of subtype-specific signature predictions in breast cancers entitled Supp_Table7.txt, it has a size of 1,001,837 bytes, and was created on Oct. 9, 2019; (8) Supplementary Table 8, Summary of Elastic Net models for gene expression signatures in lung cancers entitled Supp_Table8.txt, it has a size of 1,199,707 bytes, and was created on Oct. 9, 2019; (9) Supplementary Table 9, Summary of Elastic Net models for gene expression signatures using FOUNDATIONONE® genomic test genes entitled Supp_Table9.txt, it has a size of 56,199 bytes, and was created on Oct. 9, 2019; (10) Supplementary Table 10, Gene expression signature scores for 1038 TCGA breast tumors entitled Supp_Table10.txt, it has a size of 6,875,948 bytes, and was created on Oct. 9, 2019; (11) Supplementary Table 11, Gene expression signature scores for 512 TCGA LUAD tumors and 498 TCGA LUSC tumors entitled Supp_Table11.txt, it has a size of 6,627,663 bytes, and was created on Oct. 9, 2019; (12) Supplementary Table 12, Gene expression signature scores for 1689 METABRIC breast tumors entitled Supp_Table12.txt, it has a size of 6,235,101 bytes, and was created on Oct. 9, 2019; (13) Supplementary Table 13. Binary mutation matrix for 972 breast tumors entitled Supp_Table13.txt, it has a size of 162,934 bytes, and was created on Oct. 9, 2019; (14) Supplementary Table 14, Summary of Pan Cancer signature predictions entitled Supp_Table14.txt, it has a size of 577,688 bytes, and was created on Oct. 9, 2019; (15) Supplementary Table 15, List of amplicon signatures entitled Supp_Table15.txt, it has a size of 2,739 bytes, and was created on Oct. 9, 2019. (16) Computer program listing entitled helper.R, it has a size of 15,697 bytes, and was created on Sep. 25, 2019. All sixteen (16) files are hereby incorporated by reference in their entireties.

### 1. FIELD

The present disclosure provides a method for generating a calculated cancer signature for a cancer-related phenotype based on copy number alterations (CNAs) in a patient sample. The calculated cancer signature may correspond to a somatic mutation, an mRNA expression signature, or a protein expression signature. The disclosure also provides a for method treating a patient using the calculated cancer phenotype. In addition, the disclosure provides a method for generating a calculated signature based on CNAs to replicate a cancer phenotype.

### 2. BACKGROUND

2.1. Introduction

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Tumorigenesis is often driven by multiple types of aberrations in DNA leading to diseases of enormous complexity and heterogeneity. The ability to dissect this heterogeneity is crucial to understanding cancer mechanisms, and for identifying patient subgroups for personalized treatments. One limitation to capture this heterogeneity lies in the characterization of disease phenotypes. With the effort of many consortiums including The Cancer Genome Atlas (TCGA), large-scale multi-platform genomic data are now available, providing an opportunity to study cancer phenotypes on a molecular level and by using multiple technology types¹⁻⁴. In particular, many gene expression signatures have been developed to define specific cancer phenotypes varying from proliferation rates to features of the tumor microenvironment⁵⁻⁷. These mRNA expression features, along with protein expression, somatic mutations, and clinical features provide a comprehensive molecular portrait of tumors. Integrating multi-platform genomic data together to elucidate the relationship between genotype and phenotype is critical to understanding genetic causes underlying tumor behaviour⁸. Building predictive models for key tumor driving phenotypes would be valuable to stratify patients for personalized treatments.

### 3. SUMMARY OF THE DISCLOSURE

The present disclosure provides a method of generating a calculated cancer signature for a sample from a patient which comprises: (a) obtaining, or having obtained, a sample from the patient; (b) measuring, or having measured, a plurality of copy number alterations (CNAs) over a plurality of locations on a plurality of chromosomes; and (c) analyzing the measured CNAs using a mathematical model based on mRNA expression data and molecular subtypes, wherein the mathematical model has been validated by at least two different statistical methods so as to generate the calculated cancer signature for the sample. In one embodiment, greater than 50 CNAs are measured, alternatively greater than 100 CNAs are measured, alternatively between about 250 and about 400 CNAs are measured. In another embodiment, greater than 400 CNAs are measured. The plurality of copy number alterations (CNAs) are obtained from whole genome sequencing (WGS), whole exome sequencing (WES), or a combination thereof.

For the methods disclosed herein, the calculated cancer signature may correspond to a somatic mutation signature. The mathematical model to prepare the somatic mutation signature may be based on 10 or more beta-coefficient values in Supplemental Table 6. Alternatively, the mathematical model may be based on 20 or more beta-coefficient values, 40 or more beta-coefficient values, 60 or more beta-coefficient values, or 100 or more beta-coefficient values. In another embodiment, the mathematical model may be based on the top 5%, top 10%, top 25%, or top 50% of the beta-coefficient values.

In another embodiment of the methods disclosed herein, the calculated cancer signature may correspond to an mRNA expression signature, which may be a signature of a breast cancer subtype. The mathematical model to prepare the breast cancer subtype signature may be based on 10 or more beta-coefficient values in Supplemental Table 4. Alternatively, the mathematical model may be based on 20 or more beta-coefficient values, 40 or more beta-coefficient values, 60 or more beta-coefficient values, or 100 or more beta-coefficient values. In another embodiment, the mathematical model may be based on the top 5%, top 10%, top 25%, or top 50% of the beta-coefficient values.

In another yet embodiment of the methods disclosed herein, the calculated cancer signature may correspond to a protein expression signature. The mathematical model to prepare the protein expression signature may be based on 10 or more beta-coefficient values in Supplemental Table 5. Alternatively, the mathematical model may be based on 20 or more beta-coefficient values, 40 or more beta-coefficient values, 60 or more beta-coefficient values, or 100 or more beta-coefficient values. In another embodiment, the mathematical model may be based on the top 5%, top 10%, top 25%, or top 50% of the beta-coefficient values.

For the methods disclosed herein, the protein expression signature may be an immunohistochemistry (IHC) signature. The IHC signature may be an estrogen receptor (ER), an epidermal growth factor receptor (EGFR), a human epidermal growth factor receptor 2 (HER2), a progesterone receptor (PR), or a retinoblastoma (RB) signature.

The calculated cancer signature may correspond to a result from a commercial vendor for cancer diagnostics. For example, the calculated cancer signature may correspond to a FoundationOne® CDX result, an MAMMAPRINT® 70-GENE recurrence score, an OncotypeDX™ recurrence score, or a Prosigna® risk of recurrence score. The calculated cancer signature may be a FoundationOne® result and the mathematical model to prepare the FoundationOne® result may be based on 10 or more beta-coefficient values in Supplemental Table 9. Alternatively, the mathematical model may be based on 20 or more beta-coefficient values, 40 or more beta-coefficient values, 60 or more beta-coefficient values, or 100 or more beta-coefficient values. In another embodiment, the mathematical model may be based on the top 5%, top 10%, top 25%, or top 50% of the beta-coefficient values.

The calculated cancer signature may be associated with mutations, substitutions, or insertions or deletions (indels) in any of the following genes: (C17orf39), (MLL), (MLL2), ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BRAF, BRCA1, BRCA2, BRD4, BRIP1, BTG1, BTG2, BTK, C11orf30, CALR, CARD11, CASP8, CBFB, CBL, CCND1, CCND2, CCND3, CCNE1, CD22, CD274, CD70, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFIl, ESR1, EZH2, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D, KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, P2RY8, Page 4 of 36 RAL-0003-01, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, SDHA, SDHB, SDHC, SDHD, SETD2, SF3B1, SGK1, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TET2, TGFBR2, TIPARP, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703.

The calculated cancer signature may be associated with a rearrangement of ALK, introns 18, 19; BCL2, 3′UTR; BCR, introns 8, 13, 14; BRAF, introns 7-10; BRCA1, introns 2, 7, 8, 12, 16, 19, 20; BRCA2, intron 2; CD74, introns 6-8; EGFR, introns 7, 15, 24-27; ETV4, introns 5, 6; ETV5, introns 6, 7; ETV6, introns 5, 6; EWSR1, introns 7-13; EZR. introns 9-11; FGFR1, intron 1, 5, 17; FGFR2, intron 1, 17; FGFR3, intron 17; KIT, intron 16; KMT2A (MLL), introns 6-11; MSH2, intron 5; MYB, intron 14; MYC, intron 1; NOTCH2, intron 26; NTRK1, introns 8-10; NTRK2, intron 12; NUTM1, intron 1; PDGFRA, introns 7, 9, 11; RAF1, introns 4-8; RARA, intron 2; RET, introns 7-11; ROS1, introns 31-35; RSPO2, intron 1; SDC4, intron 2; SLC34A2, intron 4; TERC, ncRNA; TERT, Promoter; or TMPRSS2, introns 1-3.

The calculated cancer signature may be a bladder urothelial carcinoma (BLCA), cervical squamous cell carcinoma and endocervical adenocarcinoma (CESC), colon adenocarcinoma (COAD), esophageal carcinoma (ESCA), glioblastoma multiforme (GBM), head and neck squamous cell carcinoma (HNSC), kidney renal clear cell carcinoma (KIRC), kidney renal papillary cell carcinoma (KIRP), acute myeloid leukemia (LAML), brain lower grade glioma (LGG), liver hepatocellular carcinoma (LIHC), lung adenocarcinoma (LUAD), lung squamous cell carcinoma (LUSC), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), stomach adenocarcinoma (STAD), testicular germ cell tumors (TGCT), thyroid carcinoma (THCA), thymoma (THYM), or uterine corpus endometrial carcinoma (UCEC) signature. The mathematical model to prepare the calculated signature is based on 10 or more beta-coefficient values in Supplemental Table 14. Alternatively, the mathematical model may be based on 20 or more beta-coefficient values, 40 or more beta-coefficient values, 60 or more beta-coefficient values, or 100 or more beta-coefficient values. In another embodiment, the mathematical model may be based on the top 5%, top 10%, top 25%, or top 50% of the beta-coefficient values.

This disclosure also provides a method for treating a cancer patient with chemotherapy comprising the steps of: determining whether the patient has a specific cancer subtype by: (a) obtaining or having obtained a biological sample from the patient; (b) performing or having performed a gene level copy number alteration (CNA) assay on the biological sample wherein copy numbers are measured over a plurality of locations on a plurality of chromosomes; (c) comparing to results of the CNA assay to a set of standards to determine if the patient has a specific cancer subtype; and (d) if the patient has a specific cancer subtype, then administering a suitable chemotherapy regimen to the cancer patient in based on the determined cancer subtype. The chemotherapy regimen may be an ongoing therapeutic intervention. The ongoing therapeutic intervention comprises discontinuing a specific treatment.

In another embodiment, the disclosure provides a method for generating a calculated cancer signature for a cancer phenotype, the method comprising: (a) receiving a plurality of gene expression signatures and subtype information for the cancer phenotype; (b) receiving a plurality of copy number alteration (CNA) data sets for the cancer phenotype; (c) analyzing the plurality of CNA data sets with an artificial intelligence algorithm to obtain a preliminary set of CNA segment level signatures for the cancer phenotype; (d) using a gene expression training set to revise the preliminary set CNA segment level signatures and obtain a final set CNA segment level signatures; and (e) using the final set CNA segment level signatures to prepare the calculated cancer signature for the cancer phenotype. The cancer phenotype may be associated with a somatic mutation, a level of mRNA expression, a level of protein expression, or an immunohistochemistry (IHC) signature.

In addition, the disclosure provides a method for generating a calculated cancer signature for a patient, the method comprising: (a) receiving copy number alteration (CNA) data for the patient; (b) receiving one or more CNA(s) signature(s) associated with a cancer phenotype, wherein the CNA signature is based on cancer expression analysis, cancer subtype information, and CNA gain/loss information; (c) processing the CNA data for patient with an algorithm utilizing the one or more CNA(s) signature(s) associated with the cancer phenotype so as to characterize the properties of the CNA data for the patient properties relative to the one or more CNA(s) signature(s); and (d) preparing a calculated cancer signature for the patient The cancer phenotype may be associated with a somatic mutation, a level of mRNA expression, a level of protein expression, or an immunohistochemistry (IHC) signature.

For the methods disclosed herein, the cancer phenotype may be associated with an adrenal gland, a bladder, a bone, a breast, a cervix, a colon, a liver, a lung, a lymph, an ovarian, a pancreas, a penis, a prostate, a rectal, a salivary gland, a skin, a spleen, a testicular, a thymus gland, a thyroid, a trachea, or a uterine cancer. In a preferred embodiment, the cancer phenotype is associated with a breast cancer.

In another embodiment disclosure provides a method for treating a subject with cancer, comprising: (i) receiving copy number alteration (CNA) data for the patient; (ii) receiving one or more CNA(s) signature(s) associated with a cancer phenotype, wherein the CNA signature is based on cancer expression analysis, cancer subtype information, and CNA gain/loss information; (iii) processing the CNA data for the patient with an algorithm utilizing the one or more CNA(s) signature(s) associated with the cancer phenotype so as to characterize the properties of the CNA data for the patient properties relative to the one or more CNA(s) signature(s); (iv) preparing the calculated cancer signature for the patient based on the characterized properties; and (b) treating the patient based on a treatment plan based on the calculated cancer signature. The treatment may be an ongoing therapeutic intervention. The ongoing therapeutic intervention may comprise discontinuing a specific treatment.

The disclosure also provides a device comprising a processor configured to process the patient CNA data and the one or more CNA(s) signature(s) associated with the cancer phenotype with the algorithm to generate the calculated cancer signature described above. A system comprising the device of claim 36 is also provided. In the addition, the disclosure provides a device of claim 36, comprising software that comprises an algorithm to compare the patient CNA data with the one or more CNA(s) signature(s) associated with the cancer phenotype.

### 4. BRIEF DESCRIPTION OF THE FIGURES

FIG. 1A-FIG. 1D Identification of gene expression signature-specific CNAs associations in breast cancer. FIG. 1A, Schematic overview of the strategy used to identify CNAs associated with gene signatures. Gain/loss indicates DNA copy number gains or losses; Pos/Neg indicates positive or negative association. FIG. 1A-1D, Linear regression analysis landscapes accounting for molecular subtype was used to identify genes positively (dark gray above the axis) or negatively (dark gray below the axis) associated with gene signatures, and Fisher's exact test was used to compare the frequency of copy number gains (light gray above the axis) or losses (light gray below the axis) for retinoblastoma gene-loss of heterozygosity (RB-LOH) ((FIG. 1B), Basal signaling (FIG. 1C), and Estrogen signaling (FIG. 1D) Gene Program signatures. Dashed lines indicate significance threshold (q=0.01). Only q values for genes significant in both analyses were plotted. Black arrowheads indicate known pathway drivers. In each figure, chromosomal boundaries are indicated by vertical black lines.

FIG. 2A-FIG. 2B Patterns of DNA CNAs and gene expression signatures in breast cancer. FIG. 2A, Heatmap showing DNA CNAs indicating gains and losses. Samples are commonly ordered on the X axis according to molecular subtype. Genes are ordered on the Y axis according to chromosomal location. FIG. 2B, Heatmap showing gene expression signatures. Samples are commonly ordered on the X axis according to molecular subtype. Gene signature scores are median centered and clustered by centroid linkage hierarchical clustering based on Pearson correlation.

FIG. 3 Patterns of associations between DNA CNAs and amplicon signatures. Genes that had a positive correlation and increased frequency of copy number gains (q<0.01) are shown in light gray and those that had a negative correlation and an increased frequency of copy number losses (q<0.01) in samples with high signature scores (top quartile) are shown in dark gray. Each amplicon signature has positive associations with its corresponding amplicon.

FIG. 4A-FIG. 4H DNA CNA-based Elastic Net prediction models for gene signatures in breast cancer. FIG. 4A, Schematic overview of the strategy used to build Elastic Net models for predicting gene expression signature levels. FIG. 4B, Area under the curve (AUC) values for 543 signatures displayed using box and whisker plots indicating the median score (horizontal line), the interquartile range (IQR, box boundaries) and 1.5 times the IQR (whiskers); the horizontal line indicates AUC=0.75, which is considered to be “highly predicable”. Three signatures are highlighted, and their feature landscapes also shown in FIG. 4F-FIG. 4H. FIG. 4C-FIG. 4E, Receiving operating characteristics (ROC) curves and corresponding AUC values of TCGA test set and Molecular Taxonomy of Breast Cancer International Consortium (METABRIC) validation set for predicting RB-LOH (FIG. 4C), Basal signaling (FIG. 4D), and Estrogen signaling (FIG. 4E). FIG. 4F-FIG. 4H, Elastic Net selected CNA segments and/or whole chromosomal arms and their coefficients for prediction models for RB-LOH (FIG. 4F), Basal signaling (FIG. 4G) and Estrogen signaling (FIG. 4H).

FIG. 5A-5D Identification of subtype-adjusted gene signature-specific CNAs in breast cancer. FIG. 5A, Schematic overview of the strategy used to identify CNAs associated with gene signatures accounting for molecular subtypes. Gain/loss indicates DNA copy number gains or losses; Pos/Neg indicates positive or negative association. FIG. 5B-FIG. 5D, Linear regression accounting for molecular subtype was used to identify genes positively (above the axis) or negatively (below the axis) associated with gene signatures, and Fisher's exact test was used to compare the frequency of copy number gains (above the axis) or losses (below the axis) for RB-LOH (FIG. 5B), Basal signaling (FIG. 5C), and Estrogen signaling (FIG. 5D) Gene Program signatures. Dashed lines indicate significance threshold (q=0.01). Only q values for genes significant in both analyses were plotted.

FIG. 6 Histogram of permuted test set AUC values. Test set AUC values from 100 permutations per each phenotype, were plotted for each highly predictable gene expression signature, clinical receptor status, somatic mutation and intrinsic molecular subtypes. vertical line indicates AUC=0.75, which is used as the threshold to define ‘highly predictable’.

FIG. 7A-FIG. 7F CNA-based Elastic Net prediction models for multiple key expression signatures and prognosis. FIG. 7A, ROC curves and corresponding AUC values of TCGA test set and METABRIC validation set for HER1-C2 signature. FIG. 7B, Selected CNA segments and/or whole chromosomal arms and their coefficients of prediction model for HER1-C2 signature. Known drivers of EGFR pathway are highlighted with black arrows. FIG. 7C-FIG. 7F, Kaplan-Meier curves of 10-year breast cancer-specific survival stratified by gene signature score (Gene Expression) and corresponding Elastic Net prediction model (DNA CNA) for RB-LOH (FIG. 7C), Basal signaling (FIG. 7D), Estrogen signaling (FIG. 7F) and HER1-C2 (FIG. 7F) signatures. Event statistics were indicated as number of events/total patients in both High and Low groups.

FIG. 8A-FIG. 8L CNA-based Elastic Net prediction models for three clinically used breast cancer assays. FIG. 8A-FIG. 8I ROC curves and Kaplan-Meier curves of 10-year breast cancer-specific survival for Oncotype DX® recurrence score (FIG. 8A-FIG. 8C), Prosigna risk of recurrence score (FIG. 8D-FIG. 8F) and MAMMAPRINT® 70-GENE recurrence score (FIG. 8G-FIG. 8I) Kaplan-Meier curves were stratified by gene signature score (Gene Expression) and corresponding Elastic Net copy number prediction model (DNA CNA). Event statistics were indicated as number of events/total patients in both High and Low groups. FIG. 8J-FIG. 8L Elastic Net selected CNA segments and/or whole chromosomal arms and their coefficients for prediction models for Oncotype DX® recurrence score (FIG. 8J), Prosigna® risk of recurrence score (k) and MAMMAPRINT® 70-GENE recurrence score (FIG. 8L).

FIG. 9A-FIG. 9I Elastic Net models predicting individual protein expression and mutation status in breast cancer. FIG. 9A Box and whisker plots indicating the median score (horizontal line), the interquartile range (IQR, box boundaries) and 1.5 times the IQR (whiskers) of AUC values for predicting protein expression of 216 proteins and phosphoproteins from TCGA RPPA arrays. Five proteins of interest are highlighted with dots. Horizontal line indicates AUC=0.75. FIG. 9B-FIG. 9D ROC curves and corresponding AUC values of TCGA test set and METABRIC validation set for predicting clinical ER status (FIG. 9B), clinical PR status (FIG. 9C), and clinical HER2 status (FIG. 9D). FIG. 9E Selected CNA segments and/or whole chromosomal arms and their coefficients of prediction model for clinical ER status. FIG. 9F Dot plots indicate AUC values for predicting individual somatic mutations. Horizontal line indicates AUC=0.75. FIG. 9G-FIG. 9H ROC curves and corresponding AUC values of TCGA training set and TCGA test set for predicting mutations of TP53 (FIG. 9G) and mutation load (FIG. 9H). FIG. 9I Selected CNA segments and/or whole chromosomal arms and their coefficients of prediction model for mutation load.

FIG. 10A-FIG. 10C Breast Cancer subtype-specific CNA-based Elastic Net prediction models for gene signatures. FIG. 10A, Box and whisker plots indicate AUC values for predicting gene signatures within Basal-like (n=185), Luminal (n=853) and Luminal A (n=556) samples; note that the plots are stratified into immune signatures (n=78) and other signatures (n=465). The horizontal line indicates AUC=0.75. FIG. 10B, ROC curves and corresponding AUC values of TCGA testing set and METABRIC validation set for predicting CD8 T cell expression signature within Basal-like samples. FIG. 10C, Selected CNA segments and/or whole chromosomal arms and their coefficients of prediction model for CD8 T cell expression signature within Basal-like samples.

FIG. 11A-FIG. 11J CNA-based Elastic Net prediction models for intrinsic and histological subtypes in breast cancer. FIG. 11A-FIG. 11E, ROC curves and corresponding AUC values for predicting Basal-like (FIG. 11A), HER2-enriched (FIG. 11B), Luminal A (FIG. 11C), and Luminal B (FIG. 11D) subtypes, and breast cancer histology IDC vs. ILC (FIG. 11E). FIG. 11F-FIG. 11J, Selected CNA segments and/or whole chromosomal arms and their coefficients of prediction model for Basal-like (FIG. 11F), HER2-enriched (FIG. 11G), Luminal A (11H) and Luminal B (FIG. 11I) subtypes, and histology (FIG. 11J). Positive weights favor ILC classification.

FIG. 12A-FIG. 12F Selected CNA landscapes of DNA-based Elastic Net prediction models for clinical receptor status and corresponding protein expressions measured by RPPA. Selected CNA segments and/or whole chromosomal arms and their coefficients of prediction model for ER IHC status (FIG. 12A), ER RPPA expression (FIG. 12B), PR IHC status (FIG. 12C), PR RPPA expression (FIG. 12D), HER2 IHC status (FIG. 12E) and HER2 RPPA expression (FIG. 12F). Models predicting the RPPA expression and IHC status for the same protein have similar landscapes.

FIG. 13 Comparison of Elastic Net model performances using predictors of all genes and Foundation One® 313 gene set. Box and whisker plots indicating the median score (horizontal line), the interquartile range (IQR, box boundaries) and 1.5 times the IQR (whiskers) of AUC values for predicting gene signatures and individual protein expressions using all genes (left data points in each column) and Foundation One® test 313 genes (right data points in each column) in breast cancer. AUC values are highly correlated between the two categories.

FIG. 14A-FIG. 14E Pan-Cancer DNA CNA-based Elastic Net prediction models for gene signatures. FIG. 14A, Line plots indicate the number of highly predictable signatures (i.e. AUC>0.75) (upper line) and highly predictable non-amplicon signatures (lower line) in each tumor type. FIG. 14B, Box and whisker plots indicate the percentage of copy number altered genes in each tumor type. FIG. 14C, Heatmap shows the predictability of each gene signature in each tumor type. Gray indicates predictable and black indicates not predictable. Tumors and gene signatures are clustered by hierarchical clustering using Euclidean distance and complete linkage. FIG. 14D and FIG. 14E, Selected CNA segments and/or whole chromosomal arms and their coefficients of the multi-tumor prediction model for Claudin-low signature and immune signature.

FIG. 15A-15F DNA CNA-based Elastic Net prediction models for gene signatures in lung cancer. FIG. 15A, Box and whisker plots indicate AUC values for predicting gene signatures in lung cancers using models built on lung cancer data (TCGA training and TCGA testing on X axis) and that built on breast cancer data (Breast cancer model prediction on X axis). Horizontal line indicates AUC=0.75. FIG. 15B, ROC curves and corresponding AUC values for predicting a TP53 status signature showing that both models built on lung cancer data and breast cancer are successful (AUC>0.75). FIG. 15C, ROC curves and corresponding AUC values for predicting lung histology, lung adenocarcinoma (LUAD) vs. lung squamous cell carcinoma (LUSC). FIG. 15D-15E, Selected CNA segments and/or whole chromosomal arms and their coefficients of prediction models built on lung cancer (FIG. 15D), and breast cancer (FIG. 15E), for a TP53 status signature show similar feature landscapes. FIG. 15F, Selected CNA segments and/or whole chromosomal arms and their coefficients of prediction model for classifying lung histology, LUAD vs. LUSC. Positive weights favor LUSC classification.

FIG. 16A-16B DNA CNA-based Elastic Net prediction for continuous RB-LOH signature score in breast cancer. FIG. 16A, Scatter plot of predicted RB-LOH signature score against observed signature score in TCGA training set, TCGA testing set and METABRIC validation set. Gray line is fitted regression line. Pearson correlations are indicated. FIG. 16B, Selected CNA segments and/or whole chromosomal arms and their coefficients of the prediction model.

FIG. 17A-17D DNA CNA-based Elastic Net models predicting key cancer phenotypes with DNA sequencing determined copy number values in breast cancer. DNA sequencing data from exome sequencing was analyzed. Receiving operating curves (ROC) and area under ROC (AUC) values for predicting key gene expression signatures (FIG. 17A), clinical assays (FIG. 17B), clinical receptor status (FIG. 17C) and molecular subtypes (FIG. 17D).

### 5. DETAILED DESCRIPTION OF THE DISCLOSURE

Those skilled in the art would recognize that there are a number of methods to obtain copy number alteration (CNA) data for use in the methods described herein. Sources of CNA data may be traditional methods including, but not limited to, fluorescent in situ hybridization (FISH), comparative genomic hybridization (CGH), array comparative genomic hybridization (aCGH), or single nucleotide polymorphism (SNP) arrays. More recently, methods to obtain CNA data for a sample have been described using whole genome sequencing (WGS), whole exome sequencing (WES), or a combination of WGS and WES. See Hehir-Kwa, et al. (2018) The clinical implementation of copy number detection in the age of next-generation sequencing, Expert Review of Molecular Diagnostics, 18:10, 907-915 for a review. There are a number of tools available to obtain CNA data from WGS or WES. Examples include ADTEx, ControlFREEC, VarScan2, and SynthEx. See, e.g., Silva et al. (2017) SynthEx: a synthetic-normal-based DNA sequencing tool for copy number alteration detection and tumor heterogeneity profiling Genome Biology 18:66; or Zare et al. (2017) An evaluation of copy number variation detection tools for cancer using whole exome sequencing data, BMC Bioinformatics (2017) 18:286. Software programs to obtain CNA data from next generation sequencers (copy number variant (CNV) callers) and their year of release include ADTex(2014), BIC-seq (2011), BreakDancer (2009), CANOES (2014), Canvas (2011), CLAMMS (2015), cn.MOPS (2012), CNVem (2013), CNVer (2010), CnvHiTSeq (2012), CNVkit (2016), CNVnator (2011), CNVrd2 (2014), CNV-seq (2009), CODEX (2015), CONIFER (2012), CONTRA (2012), Control-FREEC (2011), CoNVaDING (2015), Copy-Seq (2010), Cortex (2012), DECoN (2016), Delly (2012), Excavator (2013), ExomeCNV (2011), ExomeCopy (2011), ExomeDepth (2012), FermiKit (2015), GASV (2009), GATK(2010), GenomeSTRiPv2 (2015), GROM-RD (2015), iCopyDAV (2018), JointSLM (2011), LUMPY (2014), Magnolya (2009), m-HMM (2013), mrCaNaVAR (2009), PEMer(2009), Pindel (2009), RDXplorer (2009), ReadDepth (2011), RSICNV (2017), Samblaster(2013), SegSeq (2009), SeqCNV (2017), SOAPsv (2011), Ulysses (2015), VariationHunter (2009), VarScan (2012), and XHMM (2012). See McCormick (Aug. 8, 2019) CNV Analysis Shifts Focus to NGS Sequences https://www.biocompare.com/Editorial-Articles/363086-CNV-Analysis-Shifts-Focus-to-NGS-Sequences/.

There are many examples of approved drugs that would benefit from the methods described herein. The drugs include small molecule kinase inhibitors such as imatinib (Gleevac®) an inhibitor of breakpoint cluster region-abelson (BCR-ABL) approved initially for chronic myelogenous leukemia (CML). Examples of monoclonal antibody kinase inhibitors are trastuzumab (Herceptin®), an inhibitor of ERB-B2 and approved for breast cancer or bevacizumab (Avastin®), an inhibitor of vascular endothelial growth factor (VEGF) approved for colorectal cancer. Other examples of drugs approved with a companion diagnostic include drugs approved for BRCA1/2 mutations, KRAS mutations and cKIT expression. Table 1 lists a number of approved drugs including a number of kinase inhibitors. See, Janne et al., 2009 Nat. Rev. Drug Disc. 8 709-723; Levitzki and Klein, 2010 Mol. Aspects Med. 31, 287-329; and Mellor et al. 2011 Tox. Sci. 120(1) 14-32; and the package inserts for the specific drugs.

TABLE 1

| | | | | Approved |
|---|---|---|---|---|
| Drug | Class | Biomarker | Intended Target(s) | Indication |
| Abemaciclib | Small | ER + HER2− | CDK 4/6 | BrCA |
| (VERZENIO ®) | molecule | | | |
| Abiraterone | Small | | 17α-hydroxylase/ | PrCA |
| (ZYTIGA ®) | molecule | | C17,20-lyase | |
| Acalabrutinib | Small | | BTK | B-cell |
| (CALQUENCE ®) | molecule | | | malignancies. |
| Ado-trastuzumab | Monoclonal | HER-2 overexpression | ERB-B2 | Metastatic BrCa |
| (KADCYLA ®) | drug | | | |
| | conjugate | | | |
| Afatinib | Small | exon 21 L858R | EGFR (ErbB1), HER2 | NSCLC |
| (GILOTRIF ®) | molecule | substitutions and exon 19 | (ErbB2), and HER4 | |
| | | deletions | (ErbB4) | |
| Alectinib | Small | ALK rearrangements | ALK | NSCLC |
| (ALECENSA ®) | molecule | | | |
| Alpelisib | Small | HER2 neg, HR pos, PI3K | PI3K | BrCA |
| (PIQRAY ®) | molecule | mutations | | |
| Atezolizumab | Monoclonal | PD-L1 expression | PD-1 | NSCLC, TNBC, |
| (TECENTRIQ ®) | | | | urothelial |
| Axitinib | Small | | VEGFR-1, VEGFR-2, | 2^{nd} line RCC |
| (INLYTA ®) | molecule | | and VEGFR-3 | |
| Bevacizumab | Monoclonal | | VEGF | Brain, cervical, |
| (AVASTIN ®) | | | | CRC, met BrCA, |
| | | | | OvCA RCC, |
| Binimetinib | Small | BRAF V600E or V600K | MEK1, MEK2 | Melanoma |
| (MEKTOVI ®) | molecule | | | |
| Cabozantinib | Small | | Multiple tyrosine | HCC, RCC |
| (CABOMETYX ®) | Molecule | | kinases including KIT, | |
| | | | AXL, MET, VEGFRs | |
| Cerinib | Small | ALK rearrangements | ALK | NSCLC |
| (ZYKADIA ®) | molecule | | | |
| Cetuximab | Monoclonal | KRAS WT, EGFR- | ERB-B1 | CRC, HNSCC |
| (ERBITUX ®) | | expressing | | |
| Cobimetinib | Small | BRAF V600E or V600K | MEK1, MEK2 | Melanoma |
| (COTELLIC ®) w/ | molecule | | | |
| Vemurafenib | | | | |
| (ZELBORAF ®) | | | | |
| Crizotinib | Small | ALK and ROS1 | EML4-ALK | NSCLC |
| (XALKORI ®) | molecule | rearrangement | rearrangements | |
| Dabrafenib | Small | BRAF V600E | BRAF | NSCLC, |
| (TAFINAR ®) | molecule | | | Melanoma |
| Dacomitinib(VIZIMPRO ® | Small | exon 21 L858R | EGFR (ErbB1), HER2 | NSCLC |
| | molecule | substitutions & exon 19 | (ErbB2), and HER4 | |
| | | deletions | (ErbB4) | |
| Dasatinib | Small | Philadelphia | ABL, ARG, KIT, | CML, ALL |
| (SPRYCEL ®) | molecule | chromosome-positive | PDGFRα/β, SRC | |
| | | (Ph+) | | |
| Enasidenib | Small | IDH-2 mutation | IDH-2 | AML |
| (IDHIFA ®) | molecule | | | |
| Encorafenib | Small | BRAF V600E or V600K | BRAF kinases | Melanoma |
| (BRAFTOVI ™) | molecule | | | |
| Entrectinib | Small | ROS1 pos or NTRK gene | TRK, ROS1, or ALK | NSCLC, NTRK |
| (ROZLYTREK ™) | molecule | fusion | | gene fusion solid |
| | | | | tumors |
| Erdafitinib | Small | FGFR2 or FGFR2 | FGFR1, FGFR2, | Adv. urothelial |
| (BALVERSA ™) | molecule | alterations | FGFR3, FGFR4 | carcinoma |
| Erlotinib | Small | exon 21 L858R | EGFR | NSCLC, |
| (TARCEVA ®) | molecule | substitutions and exon 19 | | pancreatic |
| | | deletions | | |
| Everolimus | Small | ER + HER2− | mTOR | Brain, BrCA, |
| (AFINITOR ®) | molecule | | | pancreatic, RCC |
| Gefitinib | Small | exon 21 L858R | EGFR | NSCLC |
| (IRESSA ®) | molecule | substitutions and exon 19 | | |
| | | deletions | | |
| Gilterinib | Small | FLT3 mutation | Multiple kinases | AML |
| (XOSPATA ®) | molecule | | including FLT3 | |
| Imatinib | Small | Ph + CML, Kit + GIST | ABL, ARG, PDGFR- | B-ALL, CEL, |
| (GLEEVEC ®) | molecule | | α/β, KIT | CML, CMML, |
| | | | | GIST, MDS/MPD |
| Ivosidenib | Small | IDH-1 mutation | IDH-1 | AML |
| (TIBSOVO ®) | molecule | | | |
| Lapatinib | Small | HER2 positive | EFGR (ERB-B1 and 2) | BrCA |
| (TYKERB ®) | molecule | | | |
| Midostaurin | Small | FLT3 mutation | KIT, PDGFRα/β, | AML |
| (RYDAPT ®) | molecule | | VEGFR2, PKC | |
| Nilotinib | Small | BCR-ABL fusion | ABL, ARG, KIT, | CML with |
| (TASIGNA ®) | molecule | | PDGFRα/β | imatinib resist. |
| | | | | and/or intolerance, |
| | | | | HNSCC |
| Niraparib | Small | | PARP-1, PARP-2 | OvCA |
| (ZEJULATM) | molecule | | | |
| Olaparib | Small | BRCA mutations | poly (ADP-ribose) | BrCA, OvCA |
| (LYNPARZA ®) | molecule | | polymerase (PARP) | |
| Osimerinib | Small | T790M, L858R, and exon | EGFR | NSCLC |
| (TAGRISSO ®) | molecule | 19 deletions | | |
| Palbociclib | Small | ER + HER2− | CDK 4/6 | BrCA |
| (IBRANCE ®) | molecule | | | |
| Panitumumab | Monoclonal | KRAS WT NRAS WT | EGFR | CRC |
| (VECTIBIX ®) | | | | |
| Pazopanib | Small | | VEGFR, PDGFRα/β, | RCC, 2^{nd} line adv |
| (VOTRIENT ®) | molecule | | and KIT | STS |
| Pembrolizumab | Monoclonal | PD-L1 expression | PD-1 | Bladder, cervical, |
| (KEYTRUDA ®) | | | | esophageal, |
| | | | | gastric, HCC, |
| | | | | HNSCC, NSCLC |
| Pemetrexed | Small | No EGFR or ALK | TYMS, DHFR, GART | Non-squamous |
| (ALIMTA ®) | molecule | mutations | | NSCLC |
| Pertuzumab | Monoclonal | HER-2 overexpression | ERB-B2 | BrCA |
| (PERJETA ®) | | | | |
| Ramucirumab | Monoclonal | EGFR or ALK mutations | VEGFR2 | CRC, HCC, |
| (CYRAMZA ®) | | | | NSCLC, |
| Regorafenib | Small | | Multi-kinase | CRC, GIST, HCC |
| (STIVARGA ®) | molecule | | | |
| Ribociclib | Small | ER + HER2− | CDK 4/6 | BrCA |
| (KISQALI ®) | molecule | | | |
| Rucaparib | Small | BRCA mutations | Poly (ADP-ribose) | Ovarian cancer |
| (RUBRACA ®) | molecule | | polymerase (PARP) | |
| Ruxolitinib | Small | | JAK1, JAK2 | MDS/MPD |
| (JAKAFI ™M) | molecule | | | |
| Sirolimus | Small | | mTOR | Transplant |
| (RAPAMUNE ®) | molecule | | | rejection |
| Sorafenib | Small | | B-RAF, VEGFRs, | RCC, HCC |
| (NEXAVAR ®) | molecule | | PDGFRα/β, FLT3, | |
| | | | KIT | |
| Sunitinib | Small | | VEGFR, PDGFR, | GIST, pancreatic, |
| (SUTENT ®) | molecule | | CSF1R, FLT3, KIT | RCC |
| Talazoparib | Small | BRCA mutations | poly (ADP-ribose) | BrCA |
| TALZENNA ®) | molecule | | polymerase (PARP) | |
| Temsirolimus | Small | | mTOR | RCC |
| (TORISEL ®) | molecule | | | |
| Trastuzumab | Monoclonal | HER-2 overexpression | ERB-B2 | BrCA, esophageal, |
| (HERCEPTIN ®) | | | | metastatic gastric |
| | | | | adenocarcinoma |
| Trastuzumab | Monoclonal | HER-2 overexpression | ERB-B2 | BrCA |
| (HERCEPTIN | | | | |
| HYLECTA ™) | | | | |
| Trametinib | Small | BRAF V600E or V600K | MEK1, MEK2 | NSCLC, |
| (MEKANIST ®) | molecule | | | Melanoma |
| Vandetanib | Small | | EGFR, VEGFR, TIE2, | Thyroid cancer |
| (CAPRELSA ®) | molecule | | | |
| Vemurafenib | Small | BRAF V600E | B-RAF | Melanoma |
| ZELBORAF ®) | molecule | | | |
| Venetoclax | Small | chromosome 17p deletion | BCL-2 | AML, CLL |
| (VENCLEXTA ®) | molecule | (tp53) | | |

Abbreviations: For gene targets see Gene Cards (http://www.genecards.org/). For indications: ALL, acute lymphoblastic leukemia; AML, acute myeloid leukemia; BrCA, breast cancer; CML, chronic myeloid leukemia; CMML, chronic myelomonocytic leukemia; CRC, colorectal cancer; GIST, gastrointestinal stromal tumor; HCC, hepatocellular carcinoma; HNSCC, head and neck squamous cell carcinoma; MDS/MPD, myelodysplastic syndrome/myeloproliferative disease; NSCLC, non-small cell lung cancer; OvCA, ovarian cancer; RCC, renal cell carcinoma; STS, soft tissue sarcoma; and TNBC, triple negative breast cancer.

Many of these drugs are approved for use with a companion diagnostic. For example, trastuzumab (Herceptin®) is approved for breast cancer over expressing ERB-B2 and cetuximab (Erbitux®) for patients with wild-type KRAS. Amado et al., 2008, J Clin Oncol 26 (10): 1626-1634; Allegra et al., 2009 J Clin Oncol 27 2091-2096. Another kinase inhibitor approved for use with a diagnostic is crizotinib (Xalkori®) approved with a fluorescent in situ hybridization (FISH) test for ALK rearrangements (Vysis LSI ALK Dual Color, Break Apart Rearrangement Probe; Abbott Molecular, Abbott Park, Ill.). Shah et al., 2011 Lancet Oncol 12 1004-1012; Shaw et al., 2009 J Clin Oncol 27 4247-4253. Vemurafenib (Zelboraf®) is approved for use in patients with BRAF V600E mutation (Cobas 4800 BRAF V600 Mutation Test, Roche Molecular Diagnostics, Pleasanton, CA). Chapman et al., 2011 NEJM 364 2507-2516. Additional details may be found at the US FDA website for companion diagnostics (https://www.fda.gov/MedicalDevices/ProductsandMedicalProcedure/InVitroDiagnostics/ucm30 1431.htm). See the Biomarker column in Table 1 for additional companion diagnostics.

The methods disclosed herein may be used as an aid in the diagnostics and treatment of a number of cancers. Once a particular cancer is diagnosed there are a variety of targeted therapies that a clinician may use to treat the patient. Non-limiting examples for bladder cancer include erdafitinib (BALVERSA™) or pembrolizumab (KEYTRUDA®) in Table 1, additional therapies include avelumab (BAVENCIO®), durvalumab (IMFINZI™), or nivolumab (OPDIVO®). Non-limiting examples for BrCA include abemaciclib (VERZENIO®), ado-trastuzumab emtansine (KADCYLA®), alpelisib (PIQRAY®), atezolizumab (TECENTRIQ®), Everolimus (AFINITOR®), lapatinib (TYKERB®), olaparib (LYNPARZA®), palbociclib (IBRANCE®), pertuzumab (PERJETA®), ribociclib (KISQALI®) or trastuzumab (HERCEPTIN®), or trastuzumab (HERCEPTIN HYLECTA™) in Table 1, additional therapies include anastrozole (ARIMIDEX®), exemestane (AROMASIN®), fulvestrant (FASLODEX®), letrozole (FEMARA®), neratinib (NERLYNX™), tamoxifen (SOLTAMOX®), or toremifene (FARESTON®). Non-limiting examples for CRC include bevacizumab (AVASTIN®), Cetuximab (ERBITUX®), panitumumab (VECTIBIX®), ramucirumab (CYRAMZA®), or regorafenib (STIVARGA®) in Table 1, additional therapies include ipilimumab (YERVOY®), nivolumab (OPDIVO®), or ziv-aflibercept (ZALTRAP®). Non-limiting examples for HCC include pembrolizumab (KEYTRUDA®), ramucirumab (CYRAMZA®), regorafenib (STIVARGA®), or sorafenib (NEXAVAR®) in Table 1, additional therapies include cabozantinib (CABOMETYX™), lenvatinib (LENVIMA®), or nivolumab (OPDIVO®). Non-limiting examples for kidney cancer include axitinib (INLYTA®), bevacizumab (AVASTIN®), cabozantinib (CABOMETYX®), Everolimus (AFINITOR®), pazopanib (VOTRIENT®), pembrolizumab (KEYTRUDA®), sorafenib (NEXAVAR®), sunitinib (SUTENT®), temsirolimus (TORISEL®) in Table 1, additional therapies include avelumab (BAVENCIO®), ipilimumab (YERVOY®), lenvatinib mesylate (LENVIMA®), or nivolumab (OPDIVO®). Non-limiting examples for leukemia include dasatinib (SPRYCEL®), enasidenib (IDHIFA®), gilteritinib (XOSPATA®), imatinib (GLEEVEC®), ivosidenib (TIBSOVO®), midostaurin (RYDAPT®), nilotinib (TASIGNA®), or venetoclax (VENCLEXTA®) in Table 1, additional therapies include alemtuzumab (CAMPATH®), blinatumomab (BLINCYTO®), bosutinib (BOSULIF®), duvelisib (COPIKTRA™), gemtuzumab ozogamicin (MYLOTARG™), glasdegib (DAURISMO™), ibrutinib (IMBRUVICA®), idelalisib (ZYDELIG®), inotuzumab ozogamicin (BESPONSA®), moxetumomab pasudotox-tdfk (LUMOXITI™), obinutuzumab (GAZYVA®), ofatumumab (ARZERRA®), ponatinib (ICLUSIG®), rituximab (RITUXAN®), rituximab and hyaluronidase human (RITUXAN HYCELA™), tagraxofusp-erzs (ELZONRIS™), tisagenlecleucel (KYMRIAH®), or tretinoin (VESANOID®). Non-limiting examples for lung cancers, e.g., NSCLC, include in Table 1 afatinib (GILORAF®), alectinib (ALECENSA®), atezolizumab (TECENTRIQ®), bevacizumab (AVASTIN®), ceritinib (LDK378/ZYKADIA®), crizotinib (XALKORI®), dabrafenib (TAFINAR®), dacomitinib (VIZIMPRO®), erlotinib (TARCEVA®), gefitinib (IRESSA®), osimertinib (TAGRISSO®), pembrolizumab (KEYTRUDA®), pemetrexed (ALIMTA®), ramucirumab (CYRAMZA®), trametinib (MEKANIST®), additional therapies include brigatinib (ALUNBRIG™), durvalumab (IMFINZI™), lorlatinib (LORBRENA®), necitumumab (PORTRAZZA™), nivolumab (OPDIVO®). Non-limiting examples for lymphoma include acalabrutinib (CALQUENCE®), pembrolizumab (KEYTRUDA®), venetoclax (VENCLEXTA®) in Table 1, additional therapies include axicabtagene ciloleucel (YESCARTA™), belinostat (BELEODAQ®), bexarotene (TARGRETIN®), bortezomib (VELCADE®), brentuximab vedotin (ADCETRIS copanlisib (ALIQOPA™), denileukin diftitox (ONTAK®), duvelisib (COPIKTRA™), Ibritumomab tiuxetan (ZEVALIN®), ibrutinib (IMBRUVICA®), idelalisib (ZYDELIG®), mogamulizumab-kpkc (POTELIGEO®), nivolumab (OPDIVO®), obinutuzumab (GAZYVA®), polatuzumab vedotin-piiq (POLIVY™), pralatrexate (FOLOTYN®), rituximab (Rituxan®), rituximab and hyaluronidase human (RITUXAN HYCELA™), romidepsin (ISTODAX®), siltuximab (SYLVANT®), tisagenlecleucel (KYMRIAH®), vorinostat (ZOLINZA®). Non-limiting examples for melanoma include alitretinoin (PANRETIN®), binimetinib (MEKTOVI®), cobimetinib (COTELLIC®), dabrafenib (TAFINAR®), encorafenib (BRAFTOVI™), pembrolizumab (KEYTRUDA®), trametinib (MEKANIST®), or vemurafenib (ZELBORAF®) in Table 1, additional therapies include avelumab (BAVENCIO®), cemiplimab-rwlc (LIBTAYO®), ipilimumab (YERVOY®), nivolumab (OPDIVO®), sonidegib (ODOMZO®), or vismodegib (ERIVEDGE®). Non-limiting examples for multiple myeloma (MM) include Bortezomib (VELCADE®), carfilzomib (KYPROLIS®), daratumumab (DARZALEX™), elotuzumab (EMPLICITI™), ixazomib (NINLARO®), panobinostat (FARYDAK®), selinexor (XPOVIO™). Non-limiting examples for prostate cancer include abiraterone acetate (ZYTIGA®) in Table 1, additional therapies include apalutamide (ERLEADA™), Cabazitaxel (JEVTANA®), darolutamide (NUBEQA®), enzalutamide (XTANDI®), radium 223 dichloride (XOFIGO®). Additional drugs that may be used for cancer treatment include Denosumab (XGEVA®), Dinutuximab (UNITUXIN™), iobenguane I 131 (AZEDRA®), Lanreotide acetate (SOMATULINE® Depot), lutetium Lu 177-dotatate (LUTATHERA®), niraparib (ZEJULA™), rucaparib camsylate (RUBRACA™), ruxolitinib phosphate (JAKAFI®), Sirolimus (RAPAMUNE®), or Talazoparib (TALZENNA®).

### 5.1. Definitions

While the following terms are believed to be well understood by one of ordinary skill in the art, the following definitions are set forth to facilitate explanation of the presently disclosed subject matter.

As used herein “area under curve” or “AUC” for a calculated signature is predictable if the AUC is greater than 0.60, or 0.65. The AUC for a calculated signature is “highly predictable” if it is 0.75 or greater. The AUC for a calculated signature may be 0.80, 0.85, 0.90, 0.95, 0.97, or greater.

As used herein, “clinical signs of cancer” means and includes any sign or indication of the existence of cancer in a subject, which sign or indication would be well known to the skilled artisan (e.g., oncologist, nurse practitioner). The clinical signs of cancer may be any symptom known to be associated with the cancer. Clinical signs of some cancers include, for example, chronic pain, nausea, vomiting, abnormal taste sensation, constipation, urinary symptoms (e.g., bladder spasm), respiratory symptoms, skin problems (e.g., pruritus, hair loss), or fever, among others.

As used herein, “remission” means and includes a period during which the symptoms of a cancer have been reduced or eliminated, as remission is ordinarily defined in the oncology art.

As used herein “serially monitoring” levels of a biomarker in a sample, refers to measuring levels of a biomarker in a sample more than once, e.g., quarterly, bimonthly, monthly, biweekly, weekly, every three days, daily, or several times per day. Serial monitoring of a level includes periodically measuring levels of biomarkers at regular intervals as deemed necessary by the skilled artisan.

The term “standard level” as used herein refers to a baseline level of a biomarker as determined in one or more normal subjects. For example, a baseline may be obtained from at least one subject and preferably is obtained from an average of subjects (e.g., n=2 to 100 or more), wherein the subject or subjects have no prior history of cancer. In the present invention, the measurement of biomarker levels may be carried out using the multiplexed copy number as described.

As used herein, “elevation” of a measured level of a biomarker relative to a standard level means that the amount or concentration of a biomarker in a sample is sufficiently greater in a subject relative to the standard to be detected by the methods described herein. For example, elevation of the measured level relative to a standard level may be any statistically significant elevation which is detectable. Such an elevation may include, but is not limited to, about a 1%, about a 10%, about a 20%, about a 40%, about an 80%, about a 2-fold, about a 4-fold, about an 8-fold, about a 20-fold, or about a 100-fold elevation, or more, relative to the standard. The term “about” as used herein, refers to a numerical value plus or minus 10% of the numerical value.

Non-limiting examples of signaling pathway modulators or chemotherapeutic agents known in the art are 5-fluorouracil; asparaginase; bevacizumab (AVASTIN®); bleomycin; campathecins; cetuximab (ERBITUX®); crizotinib (XALKORI®); cyclophosphamide; cytarabine; dacarbazine; dactinomycin; dasatinib (SPRYCEL®); daunorubicin; DNA methyltransferase inhibitors (DNMTs) such as azacitidine (VIDAZA®) and decitabine; doxorubicin; doxorubicin; epirubicin; erbstatin; erlotinib (TARCEVA®); estramustine; etoposide; etoposide; gefitinib (IRESSA®), gemcitabine, genistein, histone acetyl transferase inhibitors (HATs); histone deacetyl transferase inhibitors (HDACs) such as belinostat, entinostat (MS-275), panobinostat, PCI-24781, romidepsin (depsipeptide, FK-228), valproic acid, vorinostat (ZOLINZA®, SAHA) or heat shock protein inhibitors, including HSP90 inhibitors such as alvespimycin (IPI-493), AT13387, AUY922 (resorcinolic isoxazole amide), CNF2024 (BIIB021), HSP990, MPC-3100, retaspimycin (IPI-504), SNX-2112, SNX-5422, STA-9090, tanespimycin (17-AAG; KOS-953), or XL888; herbimycin A; hexamethylmelamine; hedgehog pathway inhibitors such as saridegib (IPL-926), vismodegib (ERIVEDGE™); hydroxyurea, idarubicin, ifosfamide, imatinib (GLEEVEC®), irinotecan, lapatinib (TYKERB lavendustin A, leucovorin, levamisole, mercaptopurine, methotrexate, mitomycin, mitoxantrone, mTOR inhibitors such as everolimus (AFINITOR®), sirolimus (RAPAMUNE®), temsirolimus (TORISEL®); nilotinib (TASIGNA®); nitrosoureas such as carmustine and lomustine; paclitaxel; panitumumab (VECTIBIX®); pazopanib (VOTRIENT®); pegaptanib (MACUGEN®); platinum compounds such as carboplatin, cisplatin, oxaplatin; plicamycin; procarbizine; proteasome inhibitors such as bortezomib (VELCADE®); ranibizumab (LUCENTIS®); sorafenib (NEXAVARC)); sunitinib (SUTENT®); taxanes such as docetaxel, paclitaxel, taxol; thioguanine; topotecan; trastuzumab (HERCEPTIN®); tyrosine kinase inhibitors; tyrphostins; vandetanib (CAPRELSA®); vemurafenib (ZELBORAFC)); vinblastine; vinca alkaloids; vincristine; or vinorelbine. In a preferred embodiment, the chemotherapeutic agent is bevacizumab (AVASTIN®), cetuximab (ERBITUX®), crizotinib (XALKORI®), dasatinib (SPRYCEL®), erlotinib (TARCEVA®), everolimus (AFINITOR®), gefitinib (IRESSA®), imatinib (GLEEVEC®), lapatinib (TYKERB®), nilotinib (TASIGNA®), panitumumab (VECTIBIX®), pazopanib (VOTRIENT®), sirolimus (RAPAMUNE®), sorafenib (NEXAVAR®), sunitinib (SUTENT®), temsirolimus (TORISEL®), trastuzumab (HERCEPTIN®), vandetanib (CAPRELSA®), or vemurafenib (ZELBORAF®). Further examples of chemotherapeutic agents may be found Table 1 above in standard publications and texts. See e.g., National Comprehensive Cancer Network (NCCN Guideline™) or Manual of Clinical Oncology, Dennis A. Casciato and Barry B. Lowitz, ed., 4th edition, Jul. 15, 2000, Little, Brown and Company, U.S.

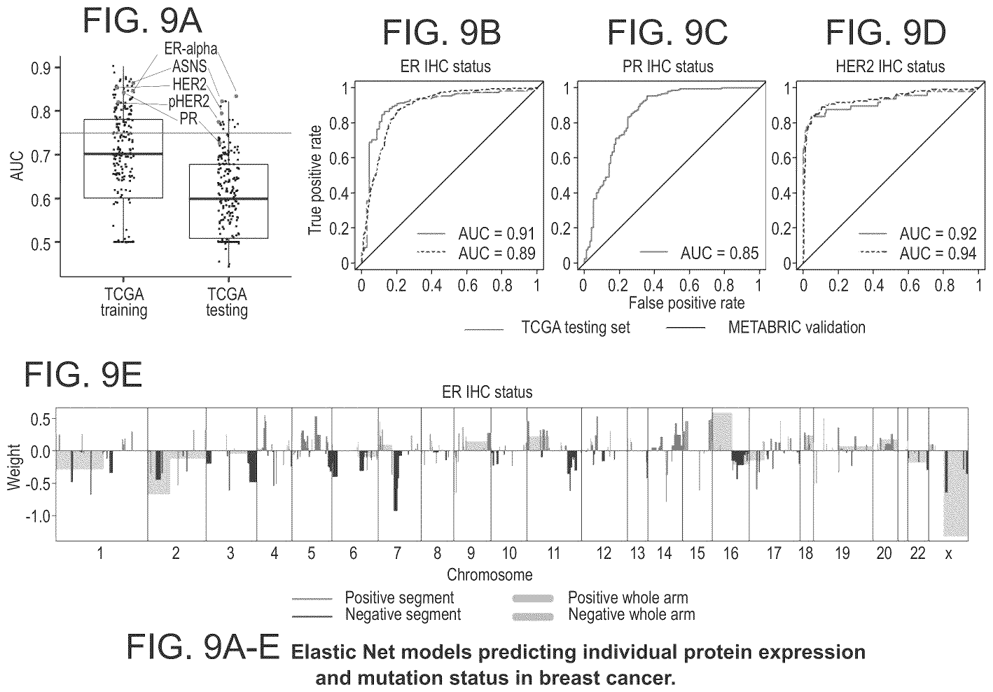

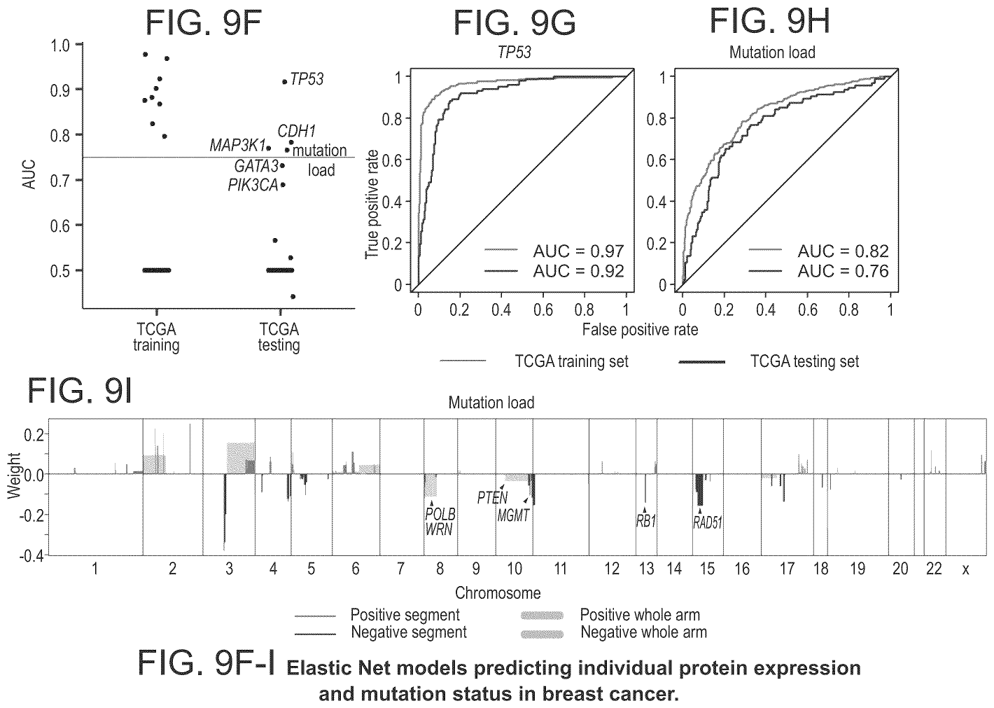

Non-limiting examples of proteins whose expression signatures may be calculated using the methods disclosed herein are: 14-3-3_zeta, 4E-BP1, 4E-BP1_pS65, 4E-BP1_pT37_T46, 4E-BP1_pT70, 53BP1, ACC_pS79, ACC1, ADAR1, Akt_pS473, Akt_pT308, AMPK_alpha, Annexin_VII, AR, A-Raf_pS299, ASNS, Bap1-c-4, Bc1-2, Bim, B-Raf, B-Raf pS445, BRD4, Caspase-8, CDK1_pY15, Chk2, Chk2_pT68, cIAP, COG3, Cyclin_B1, Cyclin_EL DJ-1, DUSP4, Dv13, eEF2K, EGFR, eIF4E, eIF4G, ER, ER-alpha, ER-alpha_pS118, ERK2, FASN, FoxM1, GAPDH, GATA3, HER2, HER2_pY1248, INPP4B, IRS1, JNK2, MSH2, MSH6, NF2, p16_INK4a, p53, p62-LCK-ligand, p70S6K, p90RSK, P-Cadherin, PCNA, PDK1, PDK1_pS241, PI3K-p110-alpha, PI3K-p85, PR, PREX1, Rab25, Rad50, Raptor, S6, Smac, Smad1, Src, VEGFR2, XRCC1, or YAP_pS127. See FIG. 9A-9E and Supplementary Table 5 for additional details and proteins.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Alternatively, depending on the context, the term “about” may mean±one half a standard deviation, ±one standard deviation, or ±two standard deviations. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-etc.). Furthermore, all values within a given range may be an endpoint for the range encompassed thereby (e.g., the range 50-80 includes the ranges with endpoints such as 55-80, 50-etc.).

As used herein, the verb “comprise” as used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

### 5.2 Computing Devices

A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a hospital or out-patient environment and communicatively connected to other computing devices (e.g., POS equipment or computers) for managing accounting, purchase transactions, and other processes within the hospital or out-patient environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 6. EXAMPLES

6.1. Summary

The ability to accurately characterize and predict tumor phenotypes, is crucial to patients for predicting prognosis. In addition, building predictive models for key phenotypes like signaling pathway activity, would be valuable to guide treatment selection. In this study, tumor DNA information and a comprehensive archive of gene expression signatures were utilized as a framework to fully characterize multiple aspects of tumor biology. An integrative computational approach using a genome-wide association analysis and an Elastic Net prediction method is presented to analyze the relationship between DNA copy number alterations and gene expression signatures. The approach was able to quantitatively predict many expression signature levels within individual tumors across breast cancers with high accuracy based upon DNA copy number features alone, including proliferation status and EGFR pathway activity. Elastic Net models were also able to predict many other key phenotypes including intrinsic molecular subtypes, some protein expression features including estrogen receptor status, and for somatic mutation status including TP53 and CDH1. This approach was successfully applied to multiple other tumor types (Pan-Cancer), which identified a number of repeatedly predictable signatures including immune cell features in squamous/basal-like cancers. These Elastic Net DNA predictors could also be called from commonly used DNA-based gene panels, thus to also inform about non-genetic tumor features that often guide therapeutic decision making. See Xia et al. (published Dec. 11, 2019) Genetic Determinants of the Molecular Portraits of Epithelial Cancers, Nat. Comm. 10:5666, the contents of which are incorporated in its entirety.

Characterization of Multiple Gene Signature-Specific DNA Copy Number Alterations

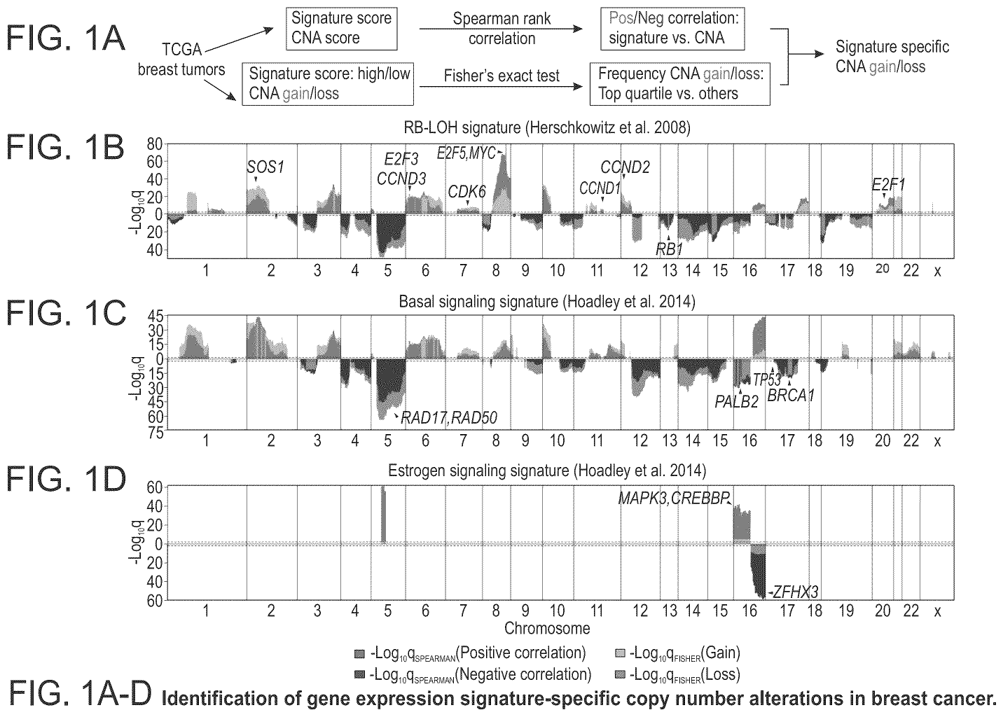

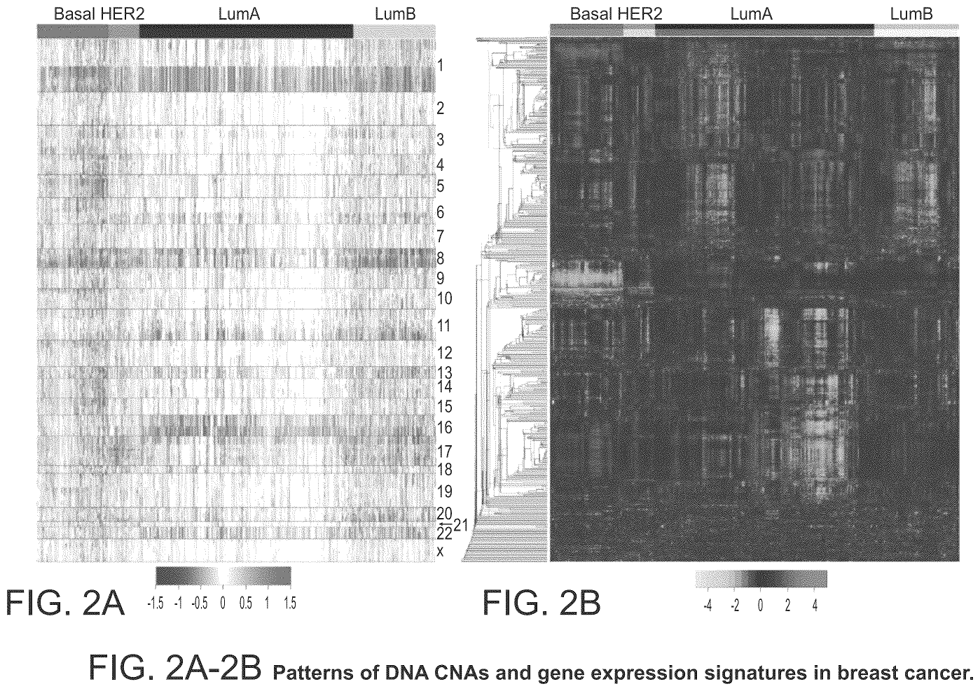

The possible associations between DNA Copy Number Alterations (CNAs) and multiple gene expression signatures was investigated first. The initial focus is on breast tumors, where multiple gene expression signatures are already in common clinical use¹⁰⁻¹². A panel of 543 published gene expression signatures¹³ measuring diverse phenotypes including multiple signaling pathways, the known prognostic/predictive models, tumor microenvironment features, and features of DNA amplicons and deletions, was applied to 1038 breast cancers using the RNA-seq data coming from the TCGA breast cancer project² (Supplementary Table 1). DNA copy number data were used to identify possible associations linking DNA CNAs to each signature-based phenotype. Previously Gatza et al.⁸ developed an association analysis method on a much smaller cohort of patients to examine the possible associations between CNAs and a limited panel of 52 gene signatures. This association analysis was modified to include another 491 signatures, and take into account molecular intrinsic subtype information (FIG. 1A) because some gene signatures showed subtype-specific associations (FIG. 2A-FIG. 2B). For each signature, two independent statistical methods were used to test for associations. Linear regression taking subtypes as covariates was used to identify positive or negative correlations between an expression signature score and gene-level DNA segment values. Following the work of Gatza et al., a Fisher's exact test was used to compare the frequency of CNA gains or losses in samples with high signature score (top quartile) and those with low signature score (all others). Both tests were Benjamini-Hochberg corrected to control the false discovery rate¹⁴. To further reduce potential false positive results, a DNA CNA feature associated with an expression signature was only called if the value was statistically significant in both analyses (q<0.01). Potential DNA CNA drivers of a signature should have positive correlations and increased copy number gains in samples with high signature scores, whereas potential repressors would have negative correlations and increased frequencies of copy number losses. Through this approach, association landscapes were analyzed for each signature, noting many expression signatures had no such associations.

The reproducibility of the association landscapes was analyzed by comparing these results to those from Gatza et al. for the same signatures⁸. All 52 signatures of Gatza et al. were included here, and in particular the RB-LOH signature¹⁵ is a focus noting that the current analysis used data on a much larger cohort of TCGA breast tumors (n=1038 vs. n=476); in addition, another systematic difference between the two studies is that Gatza et al. used gene expression microarrays while mRNA-seq was used. More importantly, molecular subtype was accounted for to identify universal associations irrespective of subtype. Despite these methodological differences, there was a high concordance between the association landscape for RB-LOH signature and that published by Gatza et al (FIG. 1B); both landscapes highlighted the identification of known RB-E2F components including DNA loss of RB1 and gains of E2F1 and E2F3, as well as the amplification of multiple cell cycle drivers including MYC and CCND2¹⁶. There were also associations present in previous results but absent in this analysis such as SOS1; this was due to the correction for intrinsic subtype as the correlation between SOS1 and RB-LOH signature was only present in Basal-like tumors.

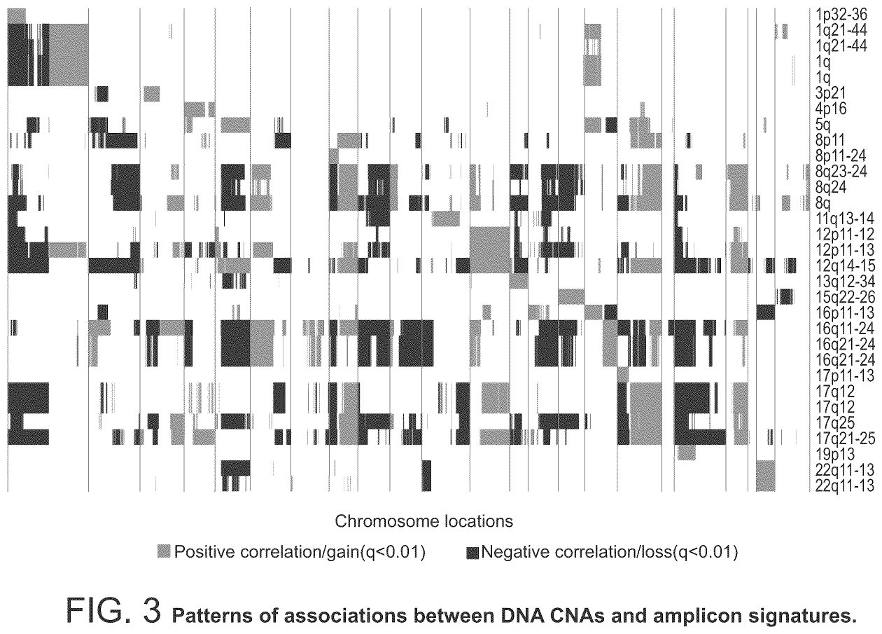

New, and old, possible associations were examined using all 543 gene signatures. Associations to previously determined DNA amplicon gene expression signatures were found and all encompassed regions of the corresponding amplicons (FIG. 3), showing that the association analysis was able to identify known DNA-based drivers of expression signatures. Two important “Gene Program” universal expression signatures defined from a 12 tumor type PanCan (n=3500) tumor analysis of Hoadley et al. 2014¹⁷, namely a “basal signaling” signature and an “estrogen signaling” signature, both showed many informative associations. For the basal signaling signature, previously known associations were identified including loss/deletion of genes involved in DNA repair such as RAD17, RAD50, PALB2 and BRCA1 (FIG. 1C). For estrogen signaling signature, many distinct luminal tumor DNA copy number changes were identified including 16p gain and 16q loss² (FIG. 1D). Collectively, these results demonstrate that the strategy disclosed herein is able to objectively find associations linking CNAs to specific gene signatures, many of which were previously known.

CNA-Based Gene Signature Predictions by Elastic Net Models

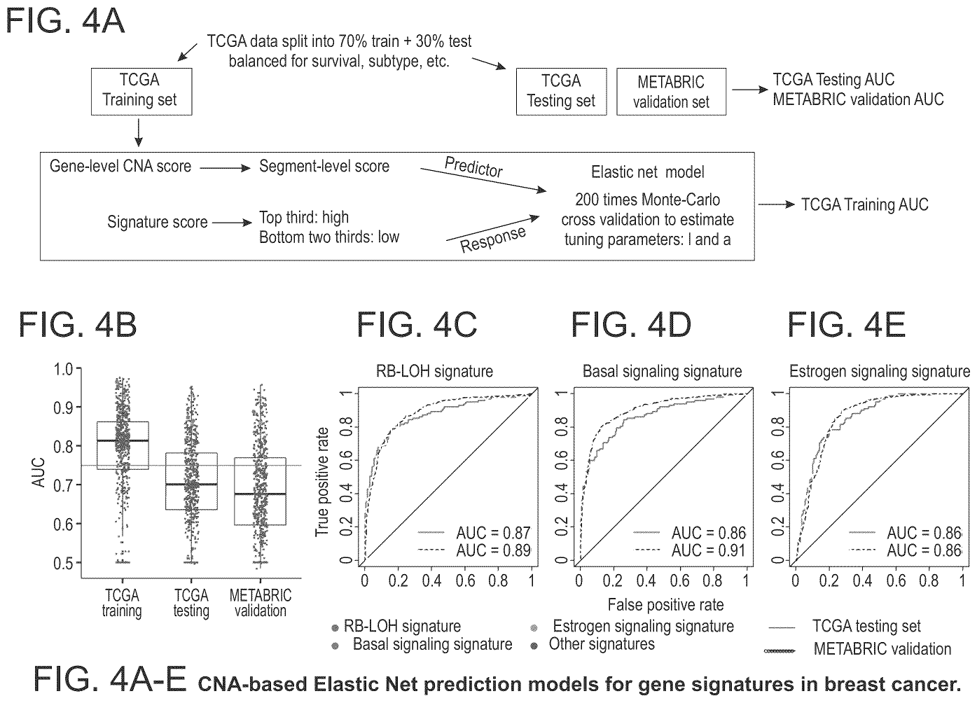

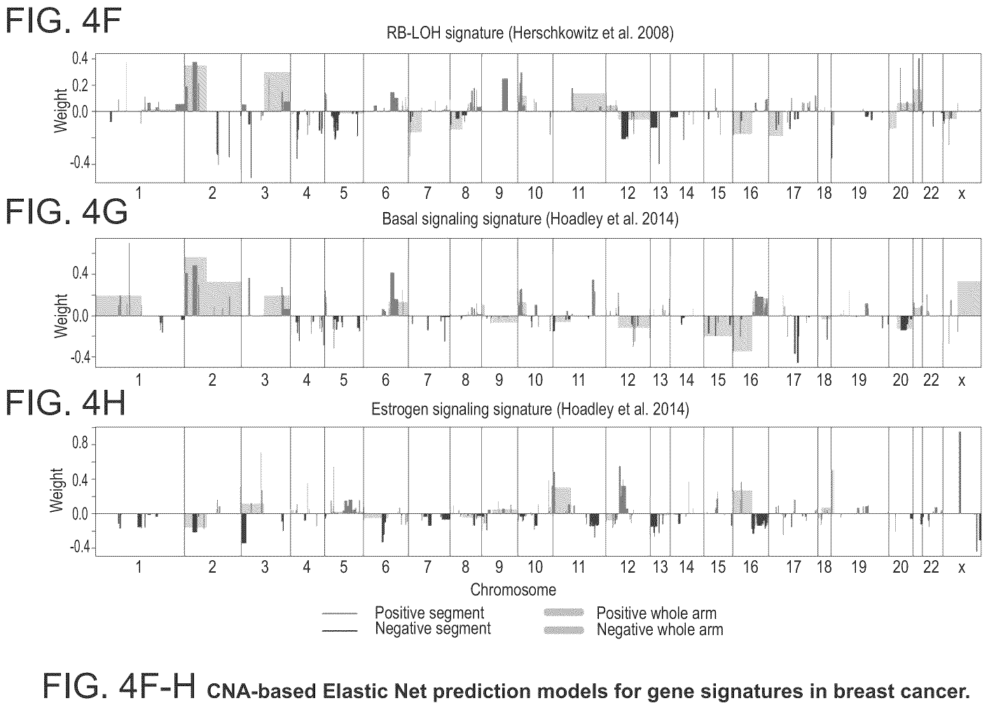

Given the strengths of these associations, the feasibility of building computational predictors of gene expression signature levels based upon DNA CNAs features only was assessed. Based on the fact that associations between CNAs and gene signatures could be found, it was possible that at least some of the expression signatures would be predictable using DNA information alone. To successfully build predictive models, a statistical modeling approach called Elastic Net was used, which is a regularized regression model that is capable of handling large numbers of potential co-linear variables and then is able to select the most relevant features to build the final model⁹. Instead of using gene-level CNA scores as predictors, 536 segment-level CNA scores were calculated using predefined chromosome regions that have been shown to be important in cancers¹⁸⁻²² (Supplementary Table 2). These DNA segments included pan-cancer significant somatic CNAs as well as breast cancer subtype-specific CNA regions. The 1038 sample TCGA breast cancer data set was split into training set (70%) and testing set (30%). Models were built solely on TCGA training set and validated on both TCGA testing set as well as a large independent breast tumor data set (Molecular Taxonomy of Breast Cancer International Consortium (METABRIC), n=1,689)³. Models were trained to classify samples into those with high signature scores (top third) versus low signature scores (bottom two-thirds). Area under ROC curve (AUC) values were used to evaluate model performance (FIG. 4A).

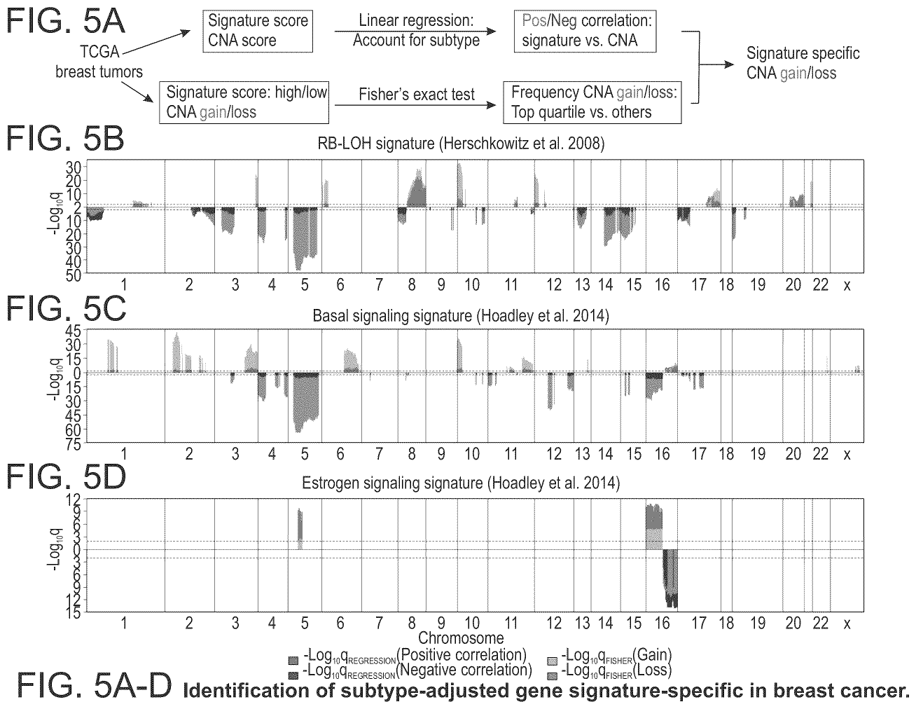

AUC distributions for all gene signatures demonstrated high predictability for some, but not all of the signatures (FIG. 4B). AUC values were of course the highest in the TCGA training set, however, AUC values were high and very similar between the TCGA testing set and METABRIC validation set for many signatures, showing that successful models were developed for multiple expression features. Of note, 142 out of the 543 signatures had AUC values above in both validation sets, determined to be “highly predictable”. Of these 142 signatures, only 33 were DNA-based amplicon signatures that essentially measure specific CNA events and were therefore expected to produce high AUC values. For example, signature 16q23-amplicon²³ had the highest AUC value in METABRIC validation set (AUC=0.96). Notably, the three signatures that highlighted for the association landscapes, namely RB-LOH signature, basal signaling signature and estrogen signaling signature, were all highly predictable (AUC>0.85) as shown by corresponding receiving operating characteristics (ROC) curves (FIG. 4C-FIG. 4E). The most predictable signatures included multiple proliferation signatures and a few oncogenic pathways, whereas the least predictable signatures were mostly those representing immune infiltrates and other features of the tumor microenvironment. In particular, a HER1-C2 signature previously developed by Hoadley et al. 2007²⁴ indicating EGFR pathway activity, had AUC values of ˜0.90 in both validation sets (FIG. 5A). Furthermore, three research-based implementations of commercially available signatures that are commonly used in the breast cancer clinic, namely OncotypeDX® recurrence score²⁵, MAMMAPRINT® 70-GENE recurrence score 26 and Prosigna® risk of recurrence score¹¹, were among the highly predictable signatures with corresponding TCGA testing set AUC values of 0.89, 0.88 and 0.91 (FIG. 5B-FIG. 5D). Permutation

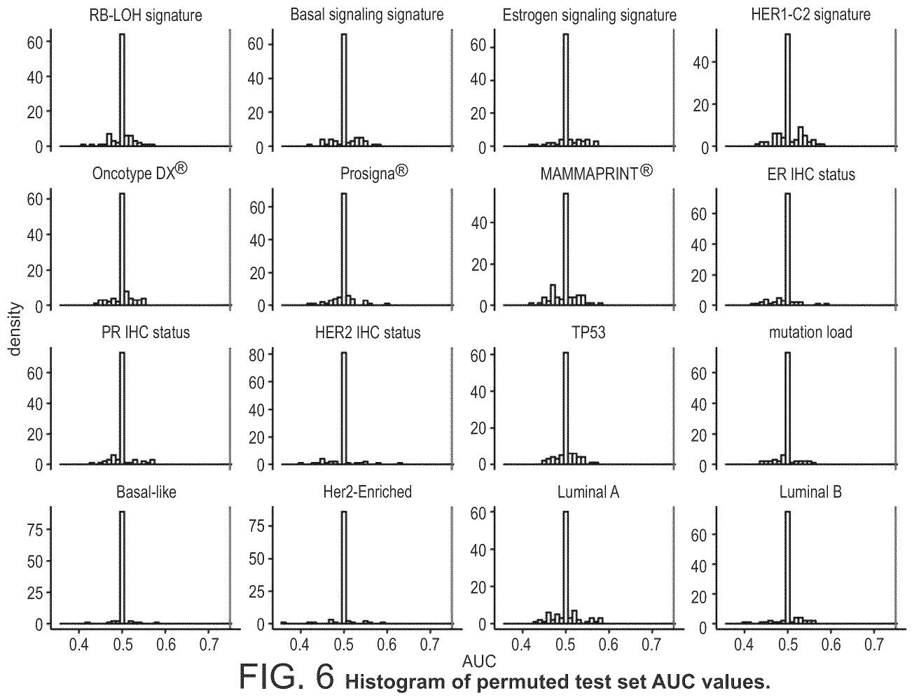

tests showed that a test set AUC of 0.75 indicates significant predictive power (FIG. 6), noting that in permuted data the highest AUC attained was 0.63 and that the large majority were close to as might be expected. Of these 142 signatures, only 33 were DNA-based amplicon signatures that essentially measure specific CNA events and were therefore expected to produce high AUC values. For example, signature 16q23-amplicon23 had the highest AUC value in METABRIC validation set (AUC=0.96).

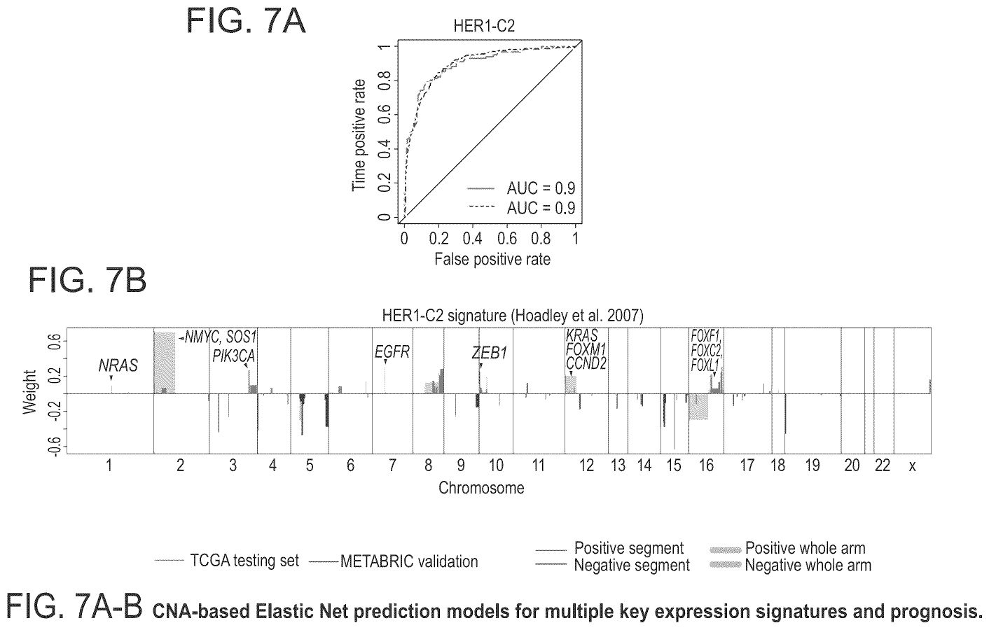

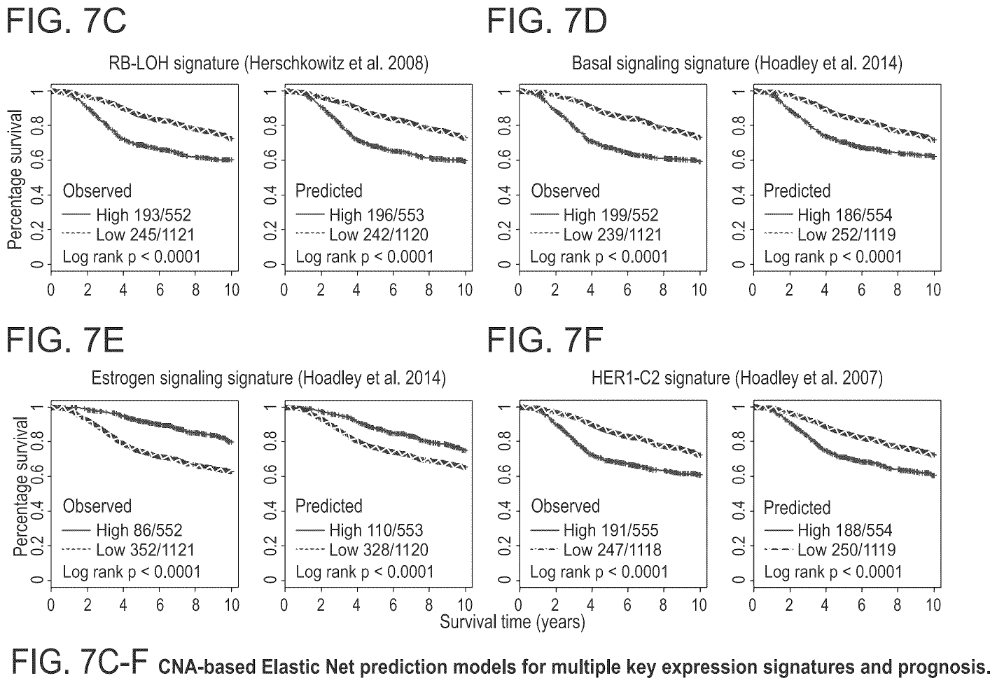

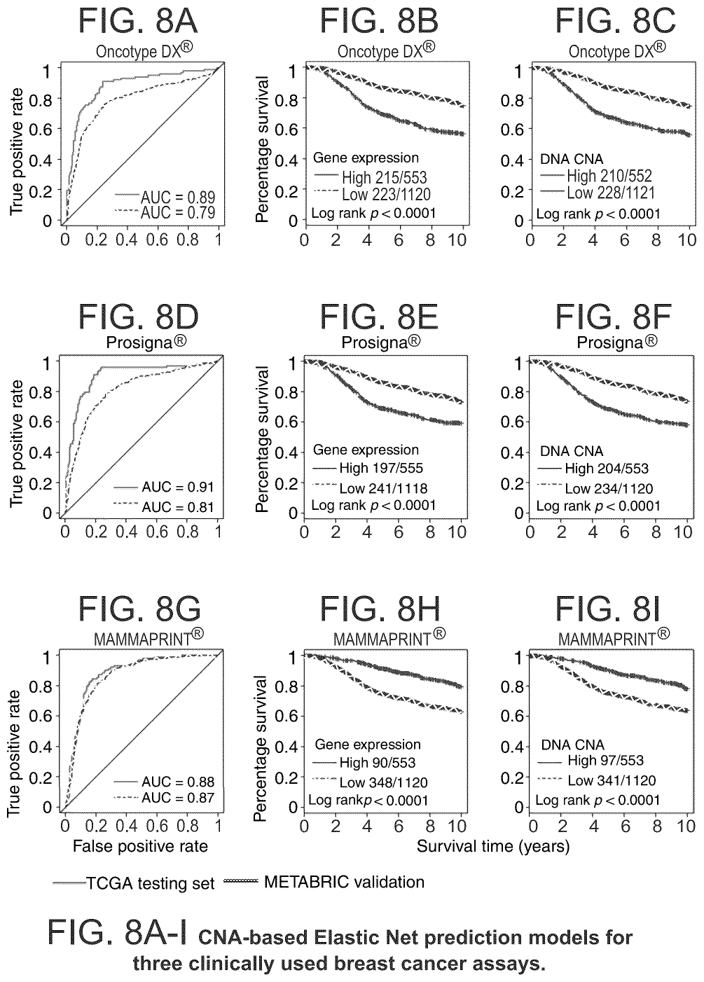

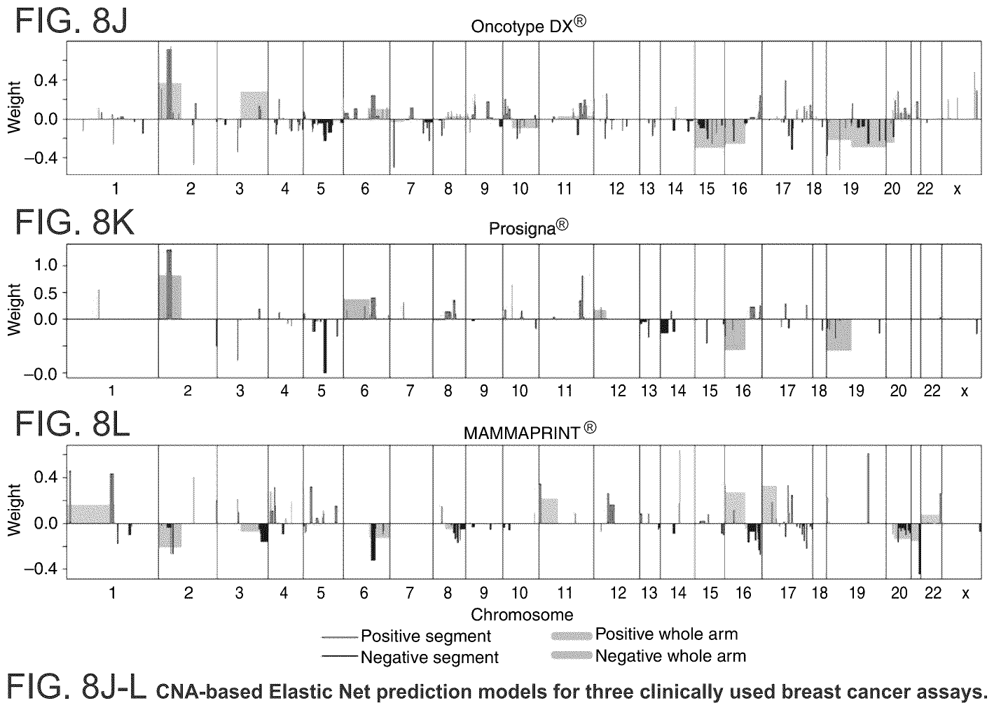

To better understand these predictive models, the CNA regions selected by the Elastic Net models (Supplementary Table 3) were investigated. To directly compare versus the association landscapes, model feature landscapes for the three signatures are shown. Remarkably, for RB-LOH signature and basal signaling signature, which had many associations with CNAs, there was a significant amount of overlap between the association landscape and the Elastic Net model feature landscape (FIG. 4F-FIG. 4G). For example, RB-E2F components as well as cell cycle components, were significantly associated with the RB-LOH signature and were selected by Elastic Net for the prediction of RB-LOH signature score. On the contrary, the estrogen signaling signature had a simple association landscape, yet the Elastic Net model selected many more features besides those in the association analysis-based regions (FIG. 4H). This suggests that Elastic Net provides additional information on the relationship between CNAs and gene signatures by taking the whole genome of data together, rather than the association analysis that evaluates genes one by one. In addition, for the highly predictable HER1-C2 signature, many EGFR pathway associated genes were selected by its Elastic Net model including EGFR itself, KRAS, SOS1 and NRAS (FIG. 7B). Taken together, these results show the ability to predict many gene expression signatures using only DNA CNAs, with high accuracy and with biological plausible and informative feature sets.

CNA-Based Predictions for Intrinsic Molecular Subtypes

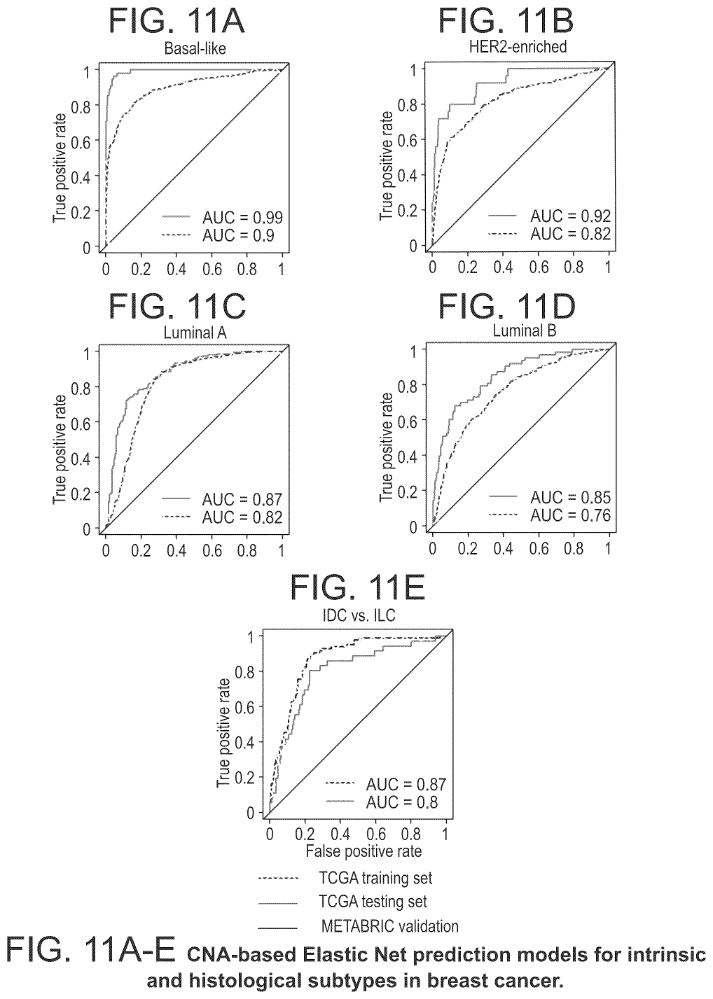

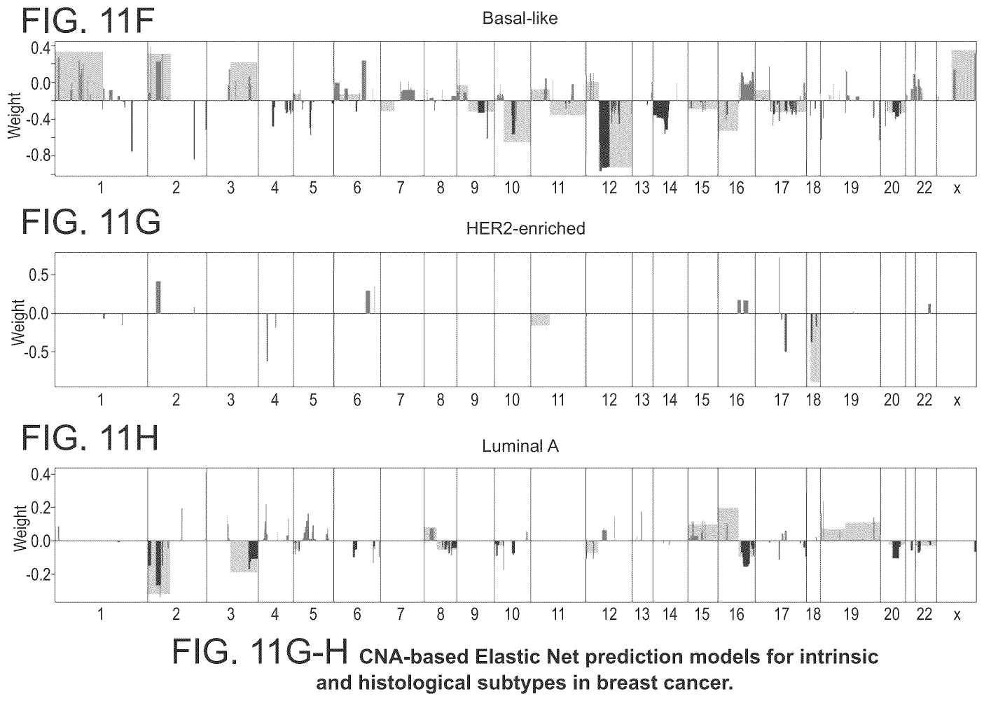

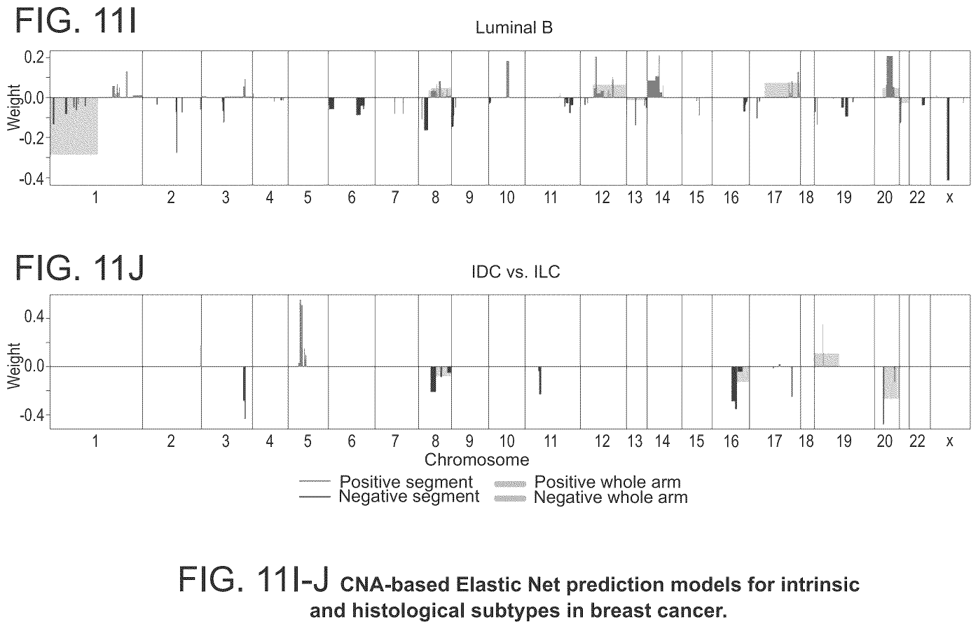

Next the Elastic Net DNA feature modeling strategy was applied to the prediction of other complex tumor phenotypes, including prediction of individual sample molecular subtypes of breast cancer¹¹. Prediction models for all intrinsic subtypes demonstrated high AUC values (i.e. >0.75) indicating that these RNA-based phenotypes can be well explained by DNA-based information (FIG. 11A-FIG. 11D, Supplementary Table 4). The Basal-like subtype had the highest test set AUC values (>0.9), consistent with previous findings that Basal-like breast cancers constitute a unique disease entity¹⁷. Regions that are frequently altered in Basal-like samples such as 1p gain and 5q loss were selected by the predictive Elastic Net model^{20,21} (FIG. 11F). HER2-Enriched subtype also had high AUC values (>0.82), and not surprisingly, regions selected by its model included the ERBB2 region, which is the dominant driver for this subtype (FIG. 11G). Luminal A and Luminal B subtypes were harder to predict, yet were still highly predictable (AUC for Luminal A=0.82, and for Luminal B=0.76 on the METABRIC validation set). A distinct difference between these two subtypes is proliferation rate where Luminal B tumors generally has higher proliferation rate than Luminal A tumors; as might be expected, regions related to proliferation including 8q(MYC) amplification and RB1 deletion were only present in the Luminal B prediction model (FIG. 11H-FIG. 11I). Since histological subtype also dictates clinical treatment decision making, how DNA CNA-based Elastic Net model predicts two major breast cancer histology was evaluated, namely invasive ductal carcinoma (IDC) and invasive lobular carcinoma (ILC). ROC curves showed high AUC values in both TCGA training set (0.87) and testing set (0.8) (FIG. 11E). A hallmark of ILC, loss of CDH1 located at chromosome 16q22.1, was reflected in the model feature landscape (FIG. 11J).

CNA-Based Predictions for Individual Protein Expression

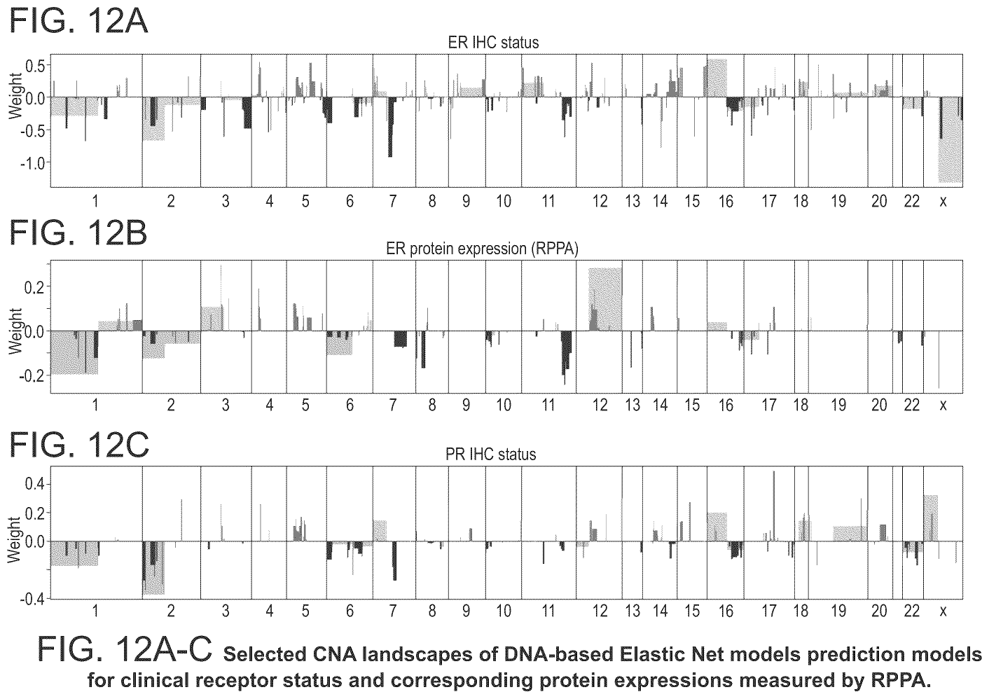

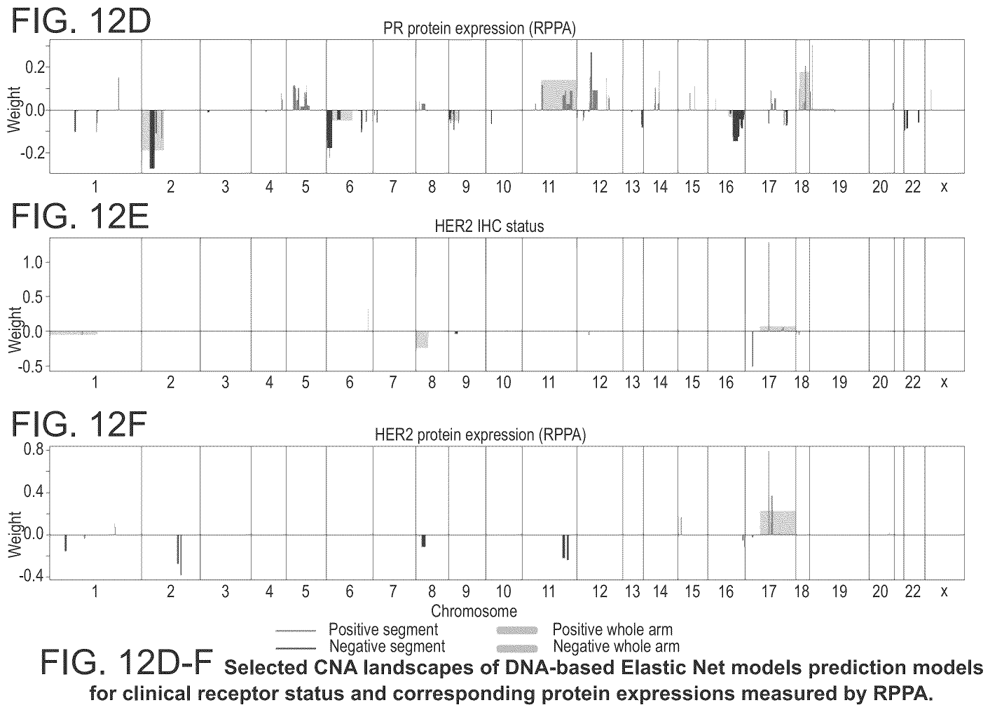

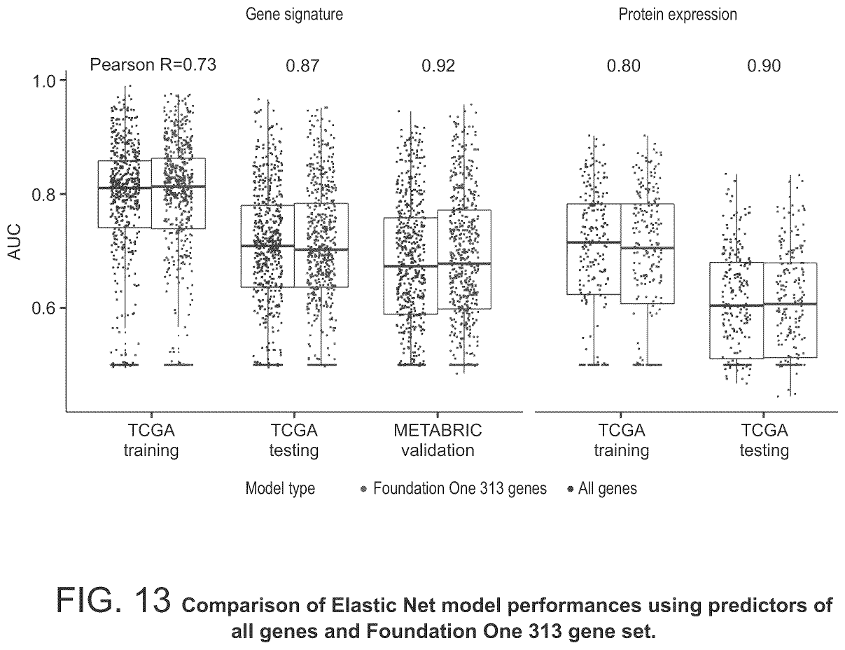

In addition, the Elastic Net DNA-based modeling strategy was applied to build prediction models for individual proteins. The reverse phase protein array (RPPA) data measuring 216 proteins and phospho-proteins coming from TCGA breast cancer samples² was utilized. Many studies have addressed the relationship between protein levels and mRNA abundance and concluded that mRNA transcript levels predict protein levels about 50% of the time²⁷. A few studies have also investigated the influence of DNA copy number on protein expression and find some proteins with significant correlations, typically those that are the target of amplification or deletion^{28,29}. However, these studies assessed correlations on individual genes. Here, the Elastic Net model is built to take into account the whole genome to predict protein expression. Using the aforementioned definition of “high precision” AUC greater or equal to 0.75, the model was able to accurately predict 16 out of the 216 protein expression levels present in the RPPA arrays (testing set AUC>0.75) (FIG. 12A-FIG. 12F, Supplementary Table 5). Clinically, in breast cancer protein expression of the estrogen receptor (ER), progesterone receptor (PR) and human epidermal growth factor receptor (HER2) direct breast cancer treatments and are routinely assessed by immunohistochemical (IHC) staining. These three proteins expression assessed by RPPA were all highly predictable by the models disclosed herein (AUC>0.75) (FIG. 9A-FIG. 9D, Supplementary Table 5). Among the other 13 highly predictable proteins, many of them were related to cell cycle including CCNB1, CCNE1 and FOXM1. Another interesting predictable protein was ASNS (testing set AUC=0.82); ASNS has recently been shown to play an important role in breast cancer metastasis, where its high expression was linked to an increased metastatic potential for lung metastases, and which represents a possible therapeutic target^{30,31}.

In breast cancer, the most critical therapeutic biomarkers are ER, PR, and HER2 scored for by immunohistochemistry. For HER2 prediction, HER2 and 17q were selected with the largest coefficients, by both the model for HER2 RPPA protein expression, and by the model guided by HER2 clinical IHC status (FIG. 12E-FIG. 12F). In contrast, protein expression of ER cannot be explained by ESRD copy number changes since ESRD copy number gain/loss is rare³². Yet the Elastic Net models were able to accurately predict ER RPPA protein expression (AUC 0.82) and ER clinical IHC status (AUC 0.89 on METABRIC validation set) when making use of DNA copy number information only. The feature landscapes of these two models were complex with many positive and negative predictors. Notably, luminal features 16p gain and 16q loss were included in the model, consistent with the fact that ER positivity is prevalent in luminal tumors (FIG. 9E). PR positivity is highly concordant with ER positivity, consistent with many regions predicting ER expression also predicted PR expression (FIG. 12A-FIG. 12D).

Finally, an increasingly common clinical assay today for cancer patients is a gene panel assay where typically hundreds of genes are DNA sequenced using massively parallel sequencing, thus giving somatic mutation status and DNA copy number values for each gene³³. One of the most widely utilized gene panels is FoundationOne®, which at the time of the writing of this manuscript contained 313 genes. Using only the DNA copy number information for these 313 genes, all gene signature and protein expression Elastic Net prediction models achieved essentially identical results and AUC values (FIG. 13); thus, these complex expression and protein phenotypes can be accurately predicted when using only a small subset of the human genome.

CNA-Based Predictions for Somatic Mutations

Next the ability to predict individual somatic mutations was examined Mutation data from TCGA breast tumors that have highly confident mutation calls 34 was utilized and the analyses was limited to the significantly mutated gene list identified by previous work as well as frequently mutated genes (frequency >5%) excluding HLA and IGH genes. Only a few mutations passed the test set AUC threshold of 0.75, namely TP53, CDH1, MAP3K1 (FIG. 9F-FIG. 9F); GATA3 and PIK3CA also had relatively high AUC values though slightly below 0.75. Both TP53 and CDH1 models selected CNA segments encompassing these two genes as negative predictors, consistent with their known tumor suppressor phenotypes. Luminal subtype specific mutation models, namely GATA3 and MAP3K1, selected luminal copy number changes including 16p gain. Interestingly, tumor mutation burden, defined here as the total number of mutations per sample that has been shown to be related to immune therapy response^{35,36}, was highly predictable from DNA CNAs (FIG. 9F).

Subtype-Specific Predictions for Gene Signatures

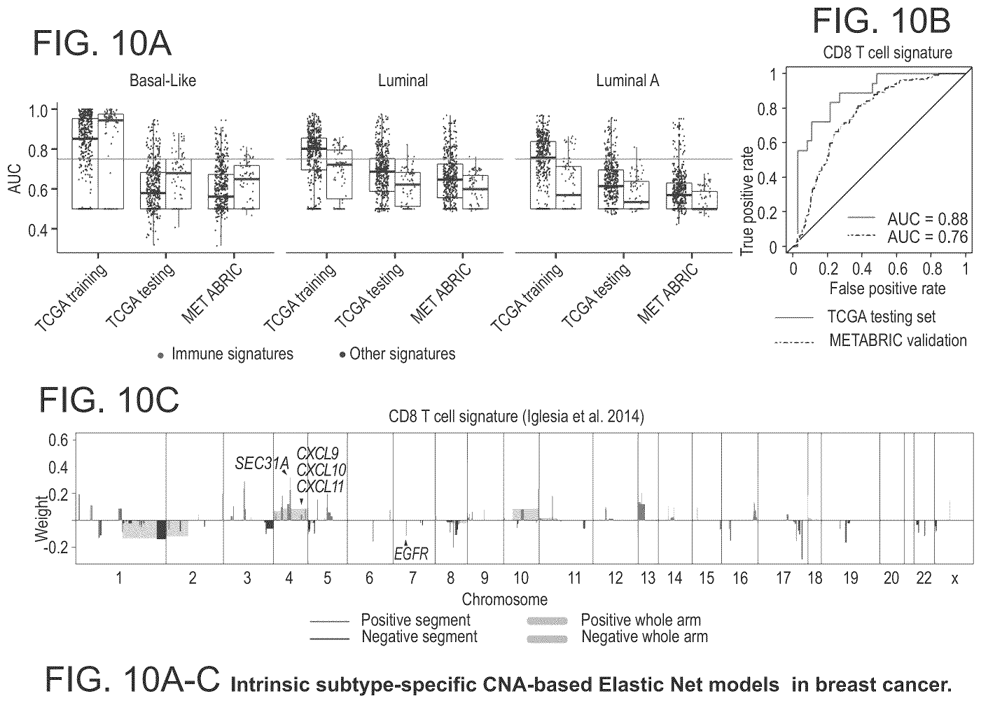

To investigate if molecular subtype affects the predictability of gene signatures, identical Elastic Net analyses was performed as described above, but only applied to Basal-like subtype tumors, Luminal A subtype tumors, or Luminal tumors (HER2-Enriched, Luminal A and Luminal B combined). Results showed that prediction accuracies differed across different subtypes (Supplementary Table 7), and in some cases, also which signatures were predicable varied. One striking phenomenon was that some immune signatures that had low AUC values using models built using all breast cancer samples, had higher AUC values when using only Basal-like tumors (FIG. 10A). For instance, a CD8 T-cell signature³⁷ had AUC values of 0.71 and when using all samples versus Basal-like samples (FIG. 10B). The segments selected to predict this signature encompassed genes encoding CD8 T-cell chemokines CXCL9, CXCL10, CXCL11 and a gene that relates to chemokine secretion SEC31A, both of which affect T-cell trafficking³⁸. Interestingly, EGFR was selected as a negative predictor, providing evidence that tumor intrinsic mechanisms shape tumor immune microenvironment³⁹ (FIG. 10C). This finding demonstrates the heterogeneity underlying different subtypes and provides insights on prioritizing Basal-like tumors for immunotherapy.

Predictions for Gene Signatures in Lung Cancer

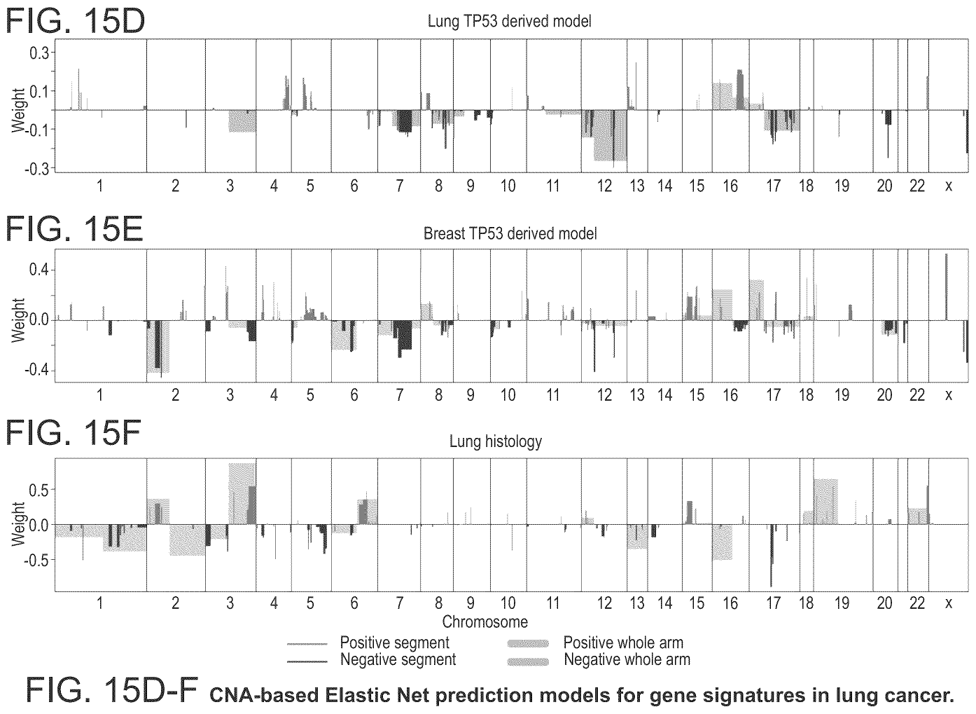

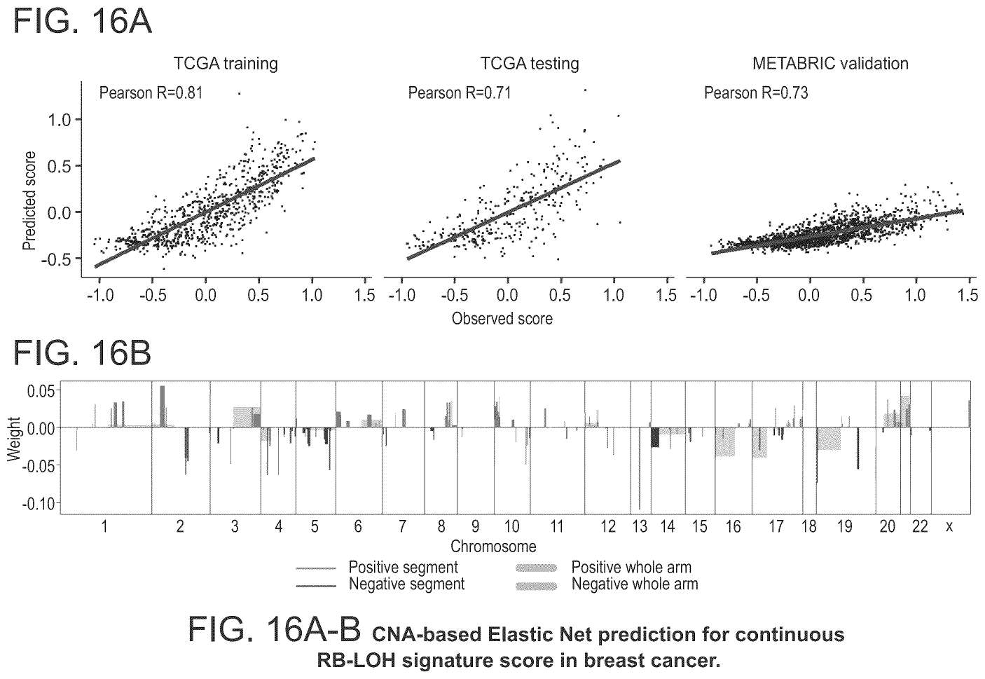

To evaluate the generalizability of the Elastic Net modeling strategy, prediction models using TCGA lung cancer data were evaluated including both lung adenocarcinoma (LUAD) and lung squamous cell carcinoma (LUSC)^{40,41}, again using gene expression signatures. First the DNA-based prediction models derived from breast cancers onto lung cancers were applied. Results identified 37 signatures that passed the AUC threshold of 0.75 across the lung training set, lung testing set and breast cancer testing set (FIG. 15A, Supplementary Table 8). Not surprisingly, most of these signatures were amplicon signatures, however, two were not and were related to TP53 mutation status and PTEN/PI3K pathway activity. CNA segments and/or whole chromosomal arms selected by models built on breast cancer or lung cancer for a TP53 status signature were similar (FIG. 15D-FIG. 15E), indicating that the Elastic Net approach was able to consistently select the most relevant features. Lastly, a larger number of signatures were found to be predictable if trained on lung cancer data, suggesting some models may be tumor type dependent, while others may be tumor type independent (i.e. TP53). The question if DNA CNA-based Elastic Net model can successfully classify the two lung cancer histologies was evaluated. Results showed that it is possible to classify LUAD vs. LUSC with very high accuracies (AUC=0.98 and 0.97 for training and testing set), consistent with previous finding that LUAD and LUSC have distinct somatic drivers⁴² (FIG. 15C, FIG. 15F).

Pan-Cancer Predictions for Gene Signatures

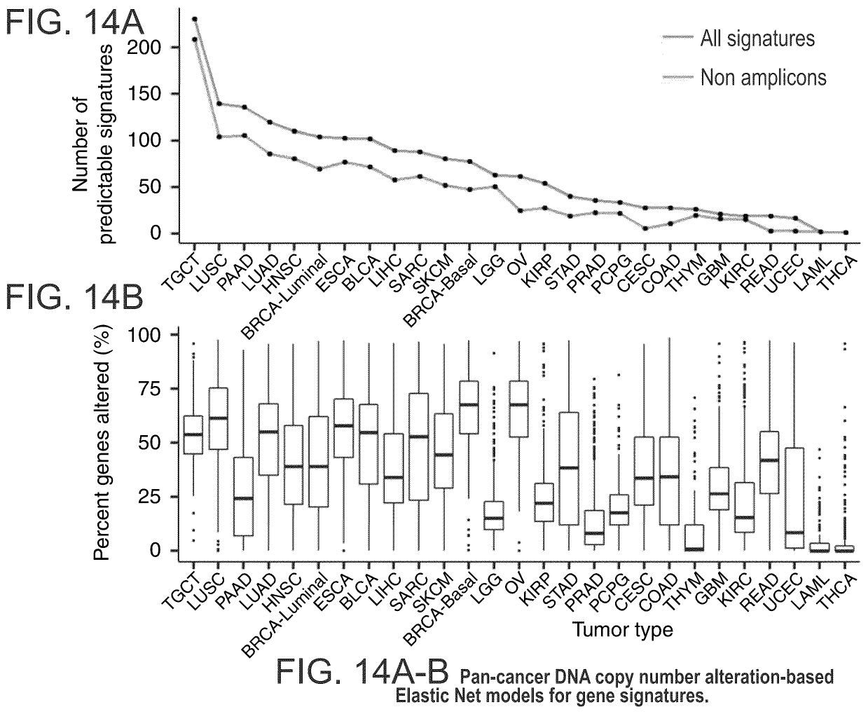

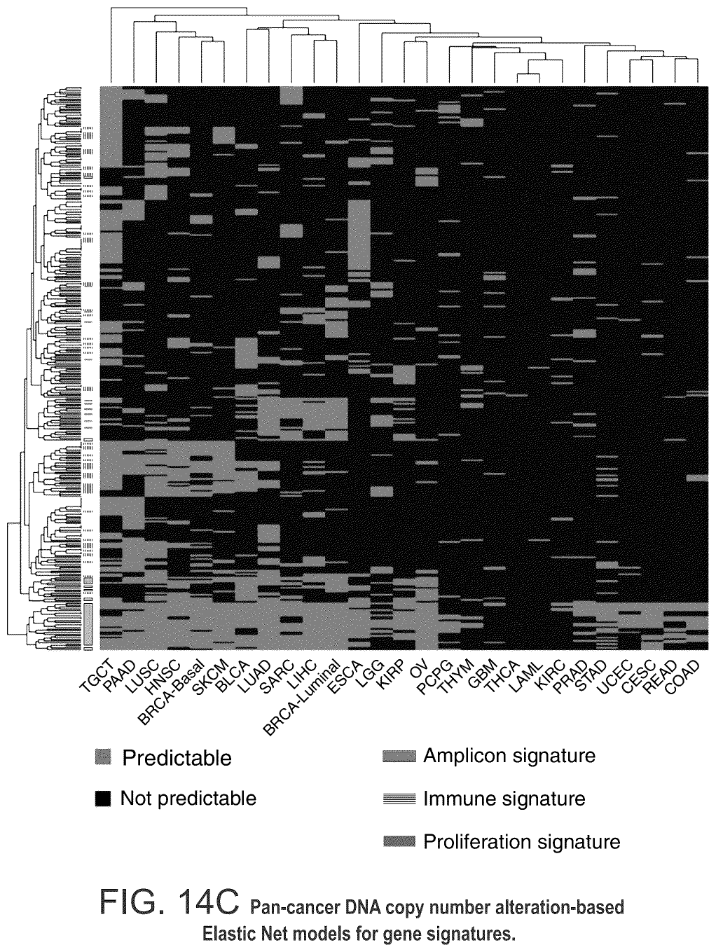

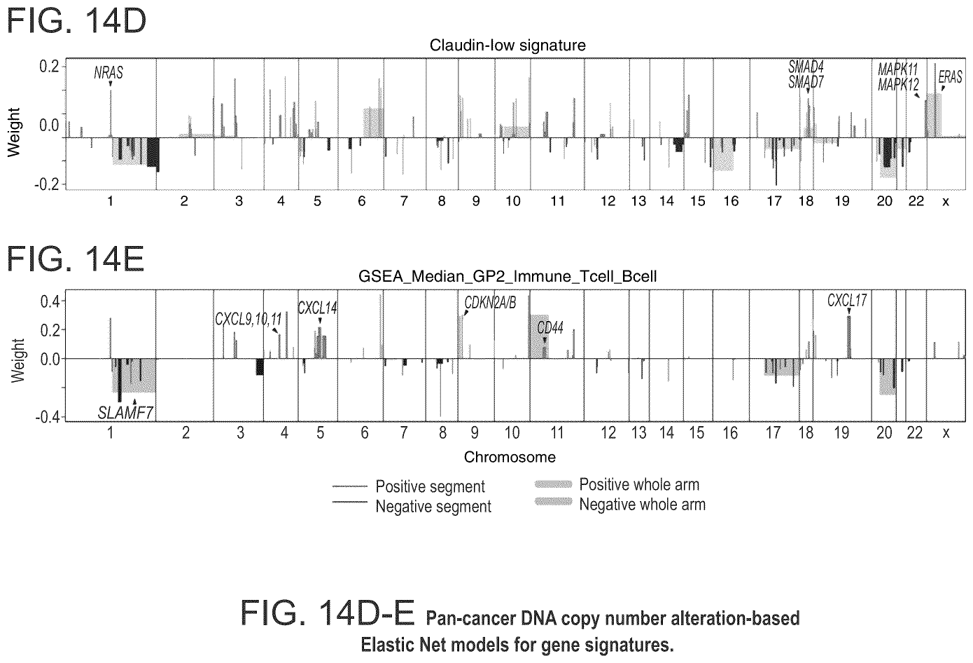

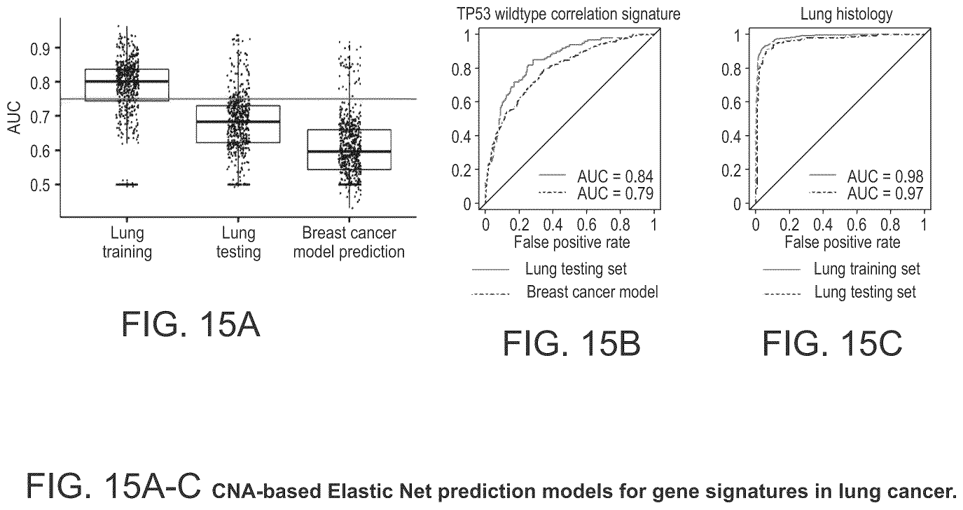

Finally the Elastic Net modeling approach was applied to 23 other tumor types from TCGA that have multi-platform data and at least 100 samples⁴. 504 median expression-based gene signatures to each tumor type were examined the predictability of each signature in each tumor type. Results showed that successful models with high accuracy (AUC>0.75) were built for multiple tumor types besides breast cancer and lung cancer. Not surprisingly, there were more gene signatures that were highly predictable in tumor types that have more CNA events (FIG. 14A, FIG. 14B). Hierarchical clustering of tumor types based on the predictability of gene signatures revealed informative tumor subgroupings consistent with previous findings on similarities between tumor types (FIG. 14C) including a Pan-Squamous group⁴. Basal-like breast cancer clustered with squamous cancers including LUSC and Head and Neck squamous cell carcinoma (HNSC). Many immune-related signatures were uniquely predictable in these tumor types including the aforementioned CD8 T-cell signature as well as PD1 and CTLA4 signaling pathways. On the other hand, Luminal breast cancer clustered with LUAD and Bladder Urothelial Carcinoma (BLCA), where multiple signatures measuring proliferation rate including the RB-LOH signature were highly predictable. Lastly, amplicon signatures were universally predictable across tumor types that have high percentage of copy number altered genes.

Noting that a Claudin-low signature, representing Claudin-low subtype and epithelial-mesenchymal transition (EMT)-like state⁴³, were highly predictable among 11 tumor types, CNA regions that are universally important in predicting this signature were built in a model on combined data from these tumor types. The resulting model had training set AUC of 0.8 and testing set AUC of 0.74, indicating the multi-tumor model was able to predict the signature across 11 tumor types. In addition, CNA regions selected by this model highlighted many RAS/MAPK pathway components including a less-known gene ERAS (FIG. 14D), consistent with the finding that its forced expression induced EMT in human mammary gland cells⁴⁴. Collectively, these results demonstrated that the Elastic Net approach was able to robustly build predictive models for key gene signatures across many tumor types.

6.2. Discussion

The ability to predict key tumor phenotypes, like mutation status or biomarker levels or complex expression phenotypes, is critical to understanding the biological complexity of solid epithelial cancers. Nowadays for breast cancer, protein expression analysis is required for ER, PR and HER2, and gene expression tests are common. For lung cancer, gene panel testing is included within the standard of care, and expression analyses (both mRNA and protein) are growing in prominence, in large part due to immunotherapy. Many solid epithelial cancers, particularly breast and lung, are thought to be at least partially DNA copy number driven because a large number of copy number events occur, and many are known genetic drivers^{45,46}. It was reasoned that many key tumor phenotypes might be predicable when using the diversity of DNA copy number changes when examining a proposed copy number driven tumor type. To address this hypothesis, an extensive archive of manually curated gene expression signatures taken from multiple publications was used to study tumor phenotypes and estimate their predictability. The relationship between DNA copy number alterations and each gene expression signature was analyzed through two means; first was a genome-wide association method, while the second was to build Elastic Net prediction models and assess their accuracy. The association study allowed us to find genes positively or negatively correlated DNA features to expression signatures by evaluating genes one by one. These two methods cooperatively produced a big picture of linkages between CNAs and gene signatures. Known associations between CNAs and gene signatures were consistently found, including gene signatures of DNA amplifications and losses, and for gene signatures of more complex phenotypes including signaling pathway activities (i.e. TP53 and EGFR), and gene signatures of cellular proliferation status; in fact, the methods were able to predict many of these signatures with very high accuracy (AUC>0.9) on a true test set that even used different gene expression and DNA copy number technologies (i.e. METABRIC). Taken together each gene signature's association landscape and Elastic Net feature landscape provides CNA regions for further investigation for potential genetic drivers. In addition, further application of the Elastic Net modeling strategy to a variety of other molecular phenotypes including molecular intrinsic subtypes, protein expression levels and somatic mutation status (including tumor mutation burden) revealed the ability to accurately predict many key phenotypes in breast cancer. These models may be clinically useful and could at least provide an orthogonal approach for calling key features like ER, PR, and HER2 status in breast cancer, and might possibly even be used by itself eventually to call key phenotypes given the growing use of DNA exomes and gene panels in the cancer clinic.

For the analyses presented here, the expression signatures were divided into the highest tertile versus the bottom two tertiles; however, Elastic Net models where the expression signatures were treated as continuous variables were also analyzed, and these were also successful for those models that showed high AUCs when tested as dichotomous variables (FIG. 12A-FIG. 12F), albeit with lower but still acceptable accuracies. Thus, it may even be feasible to predict quantitative traits, in addition to the simple high versus low as was done for the majority of the predictors.

Many commercial gene panel tests have been developed with the goal of improving precision medicine. Using DNA CNA information of only 313 genes that can be derived by FoundationOne® genomic testing, gene expression signature prediction and protein expression prediction accuracies remained the same when compared to that using whole exome of CNA values. The 313 genes have been selected as highly cancer relevant and reported to be important in tumorigenesis. This result suggests a small part of the genome accounts for a large part of the predictive power of cancer phenotypes seen in some solid epithelial cancer types. This also sheds light on the application of Elastic Net models in the clinic. For example, various proliferation signatures, including the RB-LOH signature evaluated here, might serve as a potential biomarker for CDK4/6 inhibitors which target the RB/E2F pathway⁴⁷. The Elastic Net model for RB-LOH signature could be used to stratify patients into those with high proliferation rates, which typically identifies those with RB loss, and for whom then a CDK4/6 inhibitor would not be recommended. Further validation is needed to confirm this specific hypothetical application, however, if validated, then a whole new set of prognostic and predictive biomarkers could be read out from existing DNA-based gene panels, thus providing more guidance for precision medicine at no additional cost.

Lastly, the generalizability of this approach was shown through a Pan-Cancer analysis of 23 different tumor types. Consistent with a working hypothesis, a variety of gene signatures besides amplicon signatures were predictable in tumor types that have many copy number changes. Tumor types that have been shown to share similar features had similar patterns of signature predictability. More importantly, those shared key features were often highly predictable such as immune features in squamous tumors and proliferation rate in adenocarcinomas.

Collectively these results demonstrate the ability to build CNA-based predictors for multiple key cancer phenotypes for breast and non-small cell lung cancer patients. While most research focuses on finding genetic drivers of tumorigenesis, the work carries important implications that critical complex tumor phenotypes can be predicted using DNA information, which could be potentially used in the clinic.

6.3. Methods

Gene expression data. Illumina HiSeq 2000 RNA sequencing data for human breast cancer and lung cancer (both Lung Adenocarcinoma and Lung Squamous Cell Carcinoma) were acquired from The Broad Institute TCGA GDAC Firehose⁴. Illumina HT-29 v3 expression data for the METABRIC project (n=1,992 samples) were acquired from the European Genome-phenome Archive at the European Bioinformatics Institute³. For TCGA breast cancer and lung cancer gene expression data, gene-level RNA-Seq reads were upper-quartile normalized and log 2 transformed, filtered to genes that were expressed in over 70% of samples, median centered and sample-wise standardized within each data set. For METABRIC microarray gene expression data, acquired data were filtered to genes that were expressed in over 70% of samples and were median centered for each gene and standardized for each sample. For both TCGA and METABRIC breast cancer data, PAM50 subtyping was applied as previously described^{2,3,11}. Gene expression data for all other tumor types were downloaded from GDC PanCanAtlas publication site. For each tumor type, gene expression data were filtered to genes that were expressed in over 70% of samples, median centered and sample-wise standardized within each tumor type.

DNA copy number data. GISTIC2 gene-level copy number data for human breast cancer and lung cancer were acquired from The Broad Institute TCGA GDAC Firehose with no further processing. For the METABRIC project, copy number segmentation data using circular binary segmentation (CBS) algorithm were acquired from the European Genome-phenome Archive³. Using Ensembl 54 (hg18) genome build, gene-level copy number score were derived through the extreme method as used in GISTIC2⁴⁸: Genes that fell completely within a CBS-identified copy number segment were assigned corresponding segment value. Genes that overlapped with multiple segments were assigned the greatest amplification or the least deletion value among the overlapped segments. Genes with no overlapping segments were excluded from further analyses. GISTIC2 gene-level copy number data for all other tumor types were downloaded from GDC PanCanAtlas publication site with no further processing.

Protein expression data. Normalized protein expression data for human breast cancer were acquired from The Broad Institute TCGA GDAC Firehose with no further processing.

Mutation data. Mutation Annotation Format (MAF) data from 2015 TCGA Lobular Breast Cancer dataset were used³⁴. MAF file was first filtered to only include the following variant classifications: Frame_Shift_Del, Frame_Shift_Ins, In_Frame_Del, In_Frame_Ins, Missense_Mutation, Nonsense_Mutation, Nonstop_Mutation, RNA, Splice_Site, Translation_Start_Site. A binary gene by sample matrix of 1 indicating any mutation and 0 indicating no mutation was then constructed based on the filtered MAF (Supplementary Table Mutation load for each sample was then determined by the total number of mutated genes in that sample.

Gene expression signatures. A panel of 543 previously published gene expression signatures were used to fully characterize cancer phenotypes. These 543 signatures were obtained from multiple publications or GSEA⁴⁹ and were partially summarized by Tanioka et al¹³. The complete list of genes in each signature and their references is shown in Supplementary Table 1. Signature scores were calculated in a manner consistent with their derivation. For 504 signatures with homogeneous expression across the genes, median expression value was used as signature score. The rest of the signatures were based on correlation to predetermined gene centroids or based on published algorithms. For correlation-based signatures, all predetermined training sets are available to download through the GitHub repository (https://github.com/xyouli/DNA-based-predictors-of-non-genetic-cancer-phenotypes). For each such signature, DWD53 was used to first merge gene expression matrix with corresponding training set and then Pearson/Spearman correlation/Euclidean distance was computed for each sample in the merged data. For several algorithm-based signatures, corresponding R code is provided to calculate each signature. See the COMPUTER PROGRAM LISTING Appendix. See All 543 signatures were applied to TCGA breast cancer and lung cancer data as well as METABRIC data (See Supplementary Tables 12-14). 504 median-expression based signatures were applied to Pan Cancer data.

Identification of gene signature-specific CNAs. To identify associations between CNAs and gene expression signatures, two independent statistical tests were used⁸ on TCGA breast cancer cohort with matched gene expression and copy number data excluding all Normal-like samples (n=1038). For each signature, each gene was tested for significant association with molecular subtype taken into account as a confounding variable: signature˜CNA+(1|Basal)+(1|HER2)+(1|LumA)+(1|LumB) Positive/negative correlation was determined by the coefficient of CNA and p-value in the model. A Fisher's exact test was used to compare either frequency of CNA gain or loss in samples with high signature score (top quartile) and those with low signature score (bottom three quartiles). For each analysis, Benjamini-Hochberg multiple testing correction was used to adjust p values for each signature across all genes. Significant threshold was set to 0.01 to identify genes that were significant in both analyses.

Building Elastic Net prediction models. An Elastic Net modeling approach, which is a regularized regression method that linearly combines the L1 and L2 penalties of the Ridge Regression and Least Absolute Shrinkage and Selection Operator (LASSO), was used to build DNA CNA-based predictors of cancer phenotypes 9. Alternatively, other methods of machine learning may be used to build the models. Examples of such other method include, but are not limited to, LASSO: Tibshirani, Robert, 1996, “Regression shrinkage and selection via the lasso.” Journal of the Royal Statistical Society: Series B (Methodological) 58.1: 267-288; RIDGE: Hoer1, Arthur E., and Robert W. Kennard, 1970, “Ridge regression: Biased estimation for nonorthogonal problems.” Technometrics 12.1: 55-67; supporter vector machine: Cortes, Corinna, and Vladimir Vapnik, 1995, “Support-vector networks.” Machine learning 20.3: 273-297; or deep learning: LeCun, Yann, Yoshua Bengio, and Geoffrey Hinton, 2015, “Deep learning.” nature 521.7553: 436. Generally, gene-level CNA scores were first collapsed to segment-level CNA scores. The complete list of genes in each segment is shown in Supplementary Table 2¹⁸⁻²². Each segment score was calculated as the mean CNA score across genes within the segment. For each cancer phenotype, total sample was split into 70% training set and 30% testing set (R package sampling) stratified by clinical variables: overall survival, gender, race, ER status, PR status and HER2 status, histological subtype, pathologic stages and molecular subtype when available. Models were built on training set only; 200 rounds of Monte-Carlo cross validation (R package caret) were used to select the tuning parameters. Lambda values were selected over a range of alphas from 0.1 to 1 by 0.1. The optimal parameter combination was determined to have the best classification accuracy. Model with the optimal parameters was then applied to testing set and other validation sets if available. Receiving operating characteristics (ROC) curves were constructed and area under ROC curve (AUC) values were used to evaluate model performances. Phenotypes with AUC values above 0.75 are considered highly predictable.

For predicting gene expression signatures, protein expression, and mutation load that had continuous scores, models were built to classify samples with high scores (top third) versus low scores (bottom two thirds). For molecular subtype, clinical receptor status, cancer histology and mutations that had binary outcomes, models were built to classify each outcome. For breast cancer gene expression signatures, Normal-like samples were excluded (n=1038) as in association tests described above. For somatic mutations, all IGH and HLA genes were removed and only genes that have mutation frequency greater than 5% and/or significantly mutated genes identified in 2015 TCGA Lobular Breast Cancer analysis³⁴ were included (supplementary Table 15).

For subtype-specific gene signature predictions, the same Elastic Net model approach was repeated within samples of a particular subtype, split into 70% training and 30% testing, and models were applied to METABRIC samples with the same subtype.

For gene signature and histology prediction using the non-small cell lung cancer data, the whole TCGA lung data set was used that combined both LUAD (n=498) and LUSC (n=512), which were split into training and testing sets balanced for clinical variables: overall survival, gender, pathological stages and histology (LUAD or LUSC). Models were built on training set and applied to testing set. Models built on TCGA breast cancer training set were also applied to the whole lung data set. Models were also built within LUAD and LUSC separately.

For Pan Cancer gene expression signature predictions, analysis was limited to tumor types with at least 100 samples that had RNA, DNA and clinical data. 504 median expression-based gene signatures were applied to each tumor type. For each signature prediction in each tumor type, total sample was split into 70% training set and 30% testing set, balanced for gender, race and overall survival. Models were then built on training set and applied to testing set to get training and testing AUC values.

To look at the features selected by each prediction model, coefficients of CNA segments were re-mapped to genes within each segment and plotted Summary of all Elastic Net models including coefficients of CNA segments and AUC values are reported in Supplementary Tables.

Data availability. The data sets generated and/or analyzed during the current study are available within the disclosure and its supplementary information tables. All raw and primary data come from TCGA and METABRIC public data repositories.

Applications to Differing Sources of Copy Number Alteration (CNA) Data.

Methods

Copy number alternation profiles determined by DNA exome sequencing were called by CNVkit for 1067 breast cancer samples from TCGA breast cancer project⁵⁰. 536 segment values were calculated as described above. Elastic Net predictions for key gene expression signatures, clinical assays, clinical receptor statuses and molecular subtypes were made by applying the segment values from exome sequencing to existing models built from SNP array copy number data as described herein. Area under receiving operating curves (AUC) values were calculated to reflect model performances.

Results

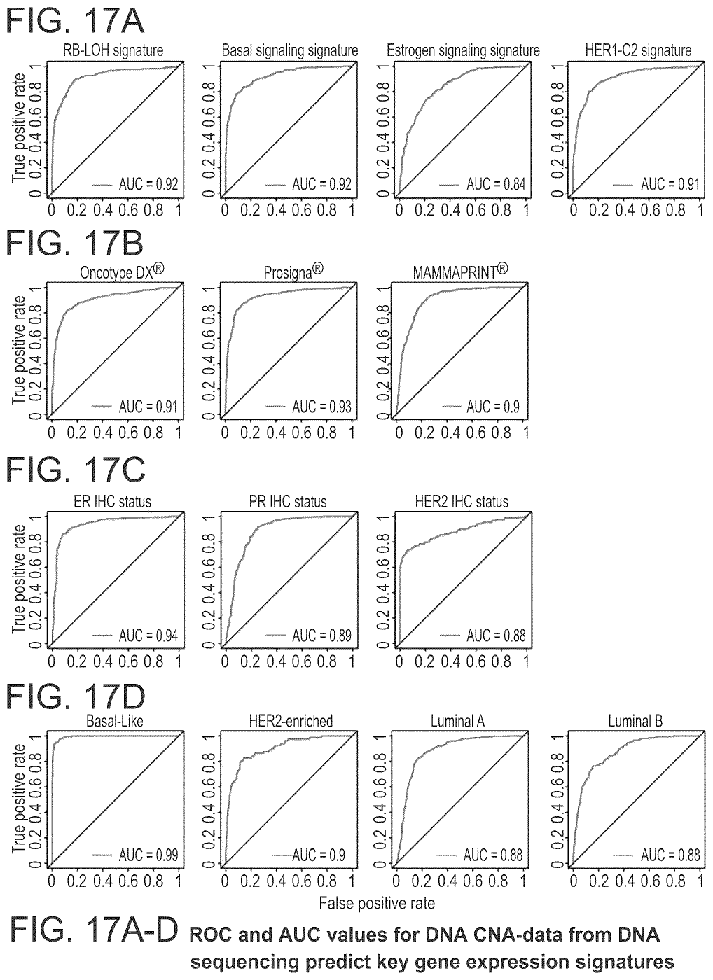

To investigate if the Elastic Net models can be called from DNA exome sequencing data, DNA exome sequencing determined copy number data was applied to models built from DNA SNP array data for representative highly predictable phenotypes. The results shown in FIG. 17A-17D demonstrate that all 14 phenotypes including four key gene expression signatures, three clinical assays, three receptor status, and four molecular subtypes, have high AUC values (>=demonstrating the applicability of the models and methods disclosed herein to DNA copy number data derived from different platforms.

### 7. REFERENCES

1 Perou, C. M. et al. Molecular portraits of human breast tumours. Nature 406, 747-752, doi:10.1038/35021093 (2000).
2 Comprehensive molecular portraits of human breast tumours. Nature 490, 61-70, doi:10.1038/nature11412 (2012).
3 Curtis, C. et al. The genomic and transcriptomic architecture of 2,000 breast tumours reveals novel subgroups. Nature 486, 346-352, doi:10.1038/nature10983 (2012).
4 Hoadley, K. A. et al. Cell-of-Origin Patterns Dominate the Molecular Classification of Tumors from 33 Types of Cancer. Cell 173, 291-304 e296, doi:10.1016/j.ce11.2018.03.022 (2018).
5 Nevins, J. R. & Potti, A. Mining gene expression profiles: expression signatures as cancer phenotypes. Nature reviews. Genetics 8, 601-609, doi:10.1038/nrg2137 (2007).
6 Bild, A. H. et al. Oncogenic pathway signatures in human cancers as a guide to targeted therapies. Nature 439, 353-357, doi:10.1038/nature04296 (2006).
7 Kwa, M., Makris, A. & Esteva, F. J. Clinical utility of gene-expression signatures in early stage breast cancer. Nat Rev Clin Oncol 14, 595-610, doi:10.1038/nrclinonc.2017.74 (2017).
8 Gatza, M. L., Silva, G. O., Parker, J. S., Fan, C. & Perou, C. M. An integrated genomics approach identifies drivers of proliferation in luminal-subtype human breast cancer. Nature genetics 46, 1051-1059, doi:10.1038/ng.3073 (2014).
9 Zou, H. & Hastie, T. Regularization and variable selection via the elastic net (vol B 67, pg 301, 2005). J Roy Stat Soc B 67, 768-768, doi:DOI 10.1111/j.1467-9868.2005.00527.x (2005).
10 Paik, S. et al. Gene expression and benefit of chemotherapy in women with node-negative, estrogen receptor-positive breast cancer. Journal of clinical oncology: official journal of the American Society of Clinical Oncology 24, 3726-3734, doi:10.1200/JCO.2005.04.7985 (2006).
11 Parker, J. S. et al. Supervised risk predictor of breast cancer based on intrinsic subtypes. Journal of clinical oncology: official journal of the American Society of Clinical Oncology 27, 1160-1167, doi:10.1200/JCO.2008.18.1370 (2009).
12 van de Vijver, M. J. et al. A gene-expression signature as a predictor of survival in breast cancer. N Engl J Med 347, 1999-2009, doi:10.1056/NEJMoa021967 (2002).
13 Tanioka, M. et al. Integrated Analysis of RNA and DNA from the Phase III Trial CALGB 40601 Identifies Predictors of Response to Trastuzumab-Based Neoadjuvant Chemotherapy in HER2-Positive Breast Cancer. Clinical cancer research: an official journal of the American Association for Cancer Research, doi:10.1158/1078-0432.CCR-17-3431 (2018).
14 Benjamini, Y. & Hochberg, Y. Controlling the False Discovery Rate—a Practical and Powerful Approach to Multiple Testing. J Roy Stat Soc B Met 57, 289-300 (1995).
15 Herschkowitz, J. I., He, X., Fan, C. & Perou, C. M. The functional loss of the retinoblastoma tumour suppressor is a common event in basal-like and luminal B breast carcinomas. Breast cancer research: BCR 10, R75, doi:10.1186/bcr2142 (2008).
16 Knudsen, E. S. & Wang, J. Y. Targeting the RB-pathway in cancer therapy. Clinical cancer research: an official journal of the American Association for Cancer Research 16, 1094-1099, doi:10.1158/1078-0432.ccr-09-0787 (2010).
17 Hoadley, K. A. et al. Multiplatform analysis of 12 cancer types reveals molecular classification within and across tissues of origin. Cell 158, 929-944, doi:10.1016/j.ce11.2014.06.049 (2014).
18 Beroukhim, R. et al. The landscape of somatic copy-number alteration across human cancers. Nature 463, 899-905, doi:10.1038/nature08822 (2010).
19 Zack, T. I. et al. Pan-cancer patterns of somatic copy number alteration. Nature genetics 1134-1140, doi:10.1038/ng.2760 (2013).
20 Silva, G. O. et al. Cross-species DNA copy number analyses identifies multiple 1q21-q23 subtype-specific driver genes for breast cancer. Breast cancer research and treatment 152, 347-356, doi:10.1007/s10549-015-3476-2 (2015).
21 Weigman, V. J. et al. Basal-like Breast cancer DNA copy number losses identify genes involved in genomic instability, response to therapy, and patient survival. Breast cancer research and treatment 133, 865-880, doi:10.1007/s10549-011-1846-y (2012).
22 Chao, H. H., He, X., Parker, J. S., Zhao, W. & Perou, C. M. Micro-scale genomic DNA copy number aberrations as another means of mutagenesis in breast cancer. PLoS One 7, e51719, doi:10.1371/journal.pone.0051719 (2012).
23 Fan, C. et al. Building prognostic models for breast cancer patients using clinical variables and hundreds of gene expression signatures. BMC Med Genomics 4, 3, doi:1755-8794-4-310.1186/1755-8794-4-3 (2011).
24 Hoadley, K. A. et al. EGFR associated expression profiles vary with breast tumor subtype. BMC Genomics 8, 258, doi:10.1186/1471-2164-8-258 (2007).
25 Paik, S. et al. A multigene assay to predict recurrence of tamoxifen-treated, node-negative breast cancer. N Engl J Med 351, 2817-2826, doi:10.1056/NEJMoa041588 (2004).
26 van't Veer, L. J. et al. Gene expression profiling predicts clinical outcome of breast cancer. Nature 415, 530-536, doi:10.1038/415530a (2002).
27 Liu, Y., Beyer, A. & Aebersold, R. On the Dependency of Cellular Protein Levels on mRNA Abundance. Cell 165, 535-550, doi:10.1016/j.ce11.2016.03.014 (2016).
28 Myhre, S. et al. Influence of DNA copy number and mRNA levels on the expression of breast cancer related proteins. Mol Oncol 7, 704-718, doi:10.1016/j.molonc.2013.02.018 (2013).
29 Geiger, T., Cox, J. & Mann, M. Proteomic changes resulting from gene copy number variations in cancer cells. PLoS genetics 6, e1001090, doi:10.1371/journal.pgen.1001090 (2010).
30 Knott, S. R. V. et al. Asparagine bioavailability governs metastasis in a model of breast cancer. Nature 554, 378-381, doi:10.1038/nature25465 (2018).
31 Balasubramanian, M. N., Butterworth, E. A. & Kilberg, M. S. Asparagine synthetase: regulation by cell stress and involvement in tumor biology. American journal of physiology. Endocrinology and metabolism 304, E789-799, doi:10.1152/ajpendo.00015.2013 (2013).
32 Horlings, H. M. et al. ESR1 gene amplification in breast cancer: a common phenomenon? Nature genetics 40, 807-808; author reply 810-802, doi:10.1038/ng0708-807 (2008).
33 Frampton, G. M. et al. Development and validation of a clinical cancer genomic profiling test based on massively parallel DNA sequencing. Nat Biotechnol 31, 1023-1031, doi:10.1038/nbt.2696 (2013).
34 Ciriello, G. et al. Comprehensive Molecular Portraits of Invasive Lobular Breast Cancer. Cell 163, 506-519, doi:10.1016/j.ce11.2015.09.033 (2015).
35 Liontos, M., Anastasiou, I., Bamias, A. & Dimopoulos, M. A. DNA damage, tumor mutational load and their impact on immune responses against cancer. Ann Transl Med 4, 264, doi:10.21037/atm.2016.07.11 (2016).
36 Van Allen, E. M. et al. Genomic correlates of response to CTLA-4 blockade in metastatic melanoma. Science 350, 207-211, doi:10.1126/science.aad0095 (2015).
37 Iglesia, M. D. et al. Genomic Analysis of Immune Cell Infiltrates Across 11 Tumor Types. J Natl Cancer Inst 108, doi:10.1093/jnci/djw144 (2016).
38 Sokol, C. L. & Luster, A. D. The chemokine system in innate immunity. Cold Spring Harb Perspect Biol 7, doi:10.1101/cshperspect.a016303 (2015).
39 Wellenstein, M. D. & de Visser, K. E. Cancer-Cell-Intrinsic Mechanisms Shaping the Tumor Immune Landscape. Immunity 48, 399-416, doi:10.1016/j.immuni.2018.03.004 (2018).
40 Cancer Genome Atlas Research, N. Comprehensive genomic characterization of squamous cell lung cancers. Nature 489, 519-525, doi:10.1038/nature11404 (2012).
41 Cancer Genome Atlas Research, N. Comprehensive molecular profiling of lung adenocarcinoma. Nature 511, 543-550, doi:10.1038/nature13385 (2014).
42 Campbell, J. D. et al. Distinct patterns of somatic genome alterations in lung adenocarcinomas and squamous cell carcinomas. Nature genetics 48, 607-616, doi:10.1038/ng.3564 (2016).
43 Herschkowitz, J. I. et al. Identification of conserved gene expression features between murine mammary carcinoma models and human breast tumors. Genome Biol 8, R76, doi:10.1186/gb-2007-8-5-r76 (2007).
44 Suarez-Cabrera, C. et al. The Ras-related gene ERAS is involved in human and murine breast cancer. Sci Rep 8, 13038, doi:10.1038/s41598-018-31326-4 (2018).
45 Bailey, M. H. et al. Comprehensive Characterization of Cancer Driver Genes and Mutations. Cell 173, 371-385 e318, doi:10.1016/j.ce11.2018.02.060 (2018).
46 Ding, L. et al. Perspective on Oncogenic Processes at the End of the Beginning of Cancer Genomics. Cell 173, 305-320 e310, doi:10.1016/j.ce11.2018.03.033 (2018).
47 Asghar, U., Witkiewicz, A. K., Turner, N. C. & Knudsen, E. S. The history and future of targeting cyclin-dependent kinases in cancer therapy. Nat Rev Drug Discov 14, 130-146, doi:10.1038/nrd4504 (2015).
48 Mermel, C. H. et al. GISTIC2.0 facilitates sensitive and confident localization of the targets of focal somatic copy-number alteration in human cancers. Genome Biol 12, R41, doi:10.1186/gb-2011-12-4-r41 (2011).
49 Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).
50 Talevich E, ShaM AH, Botton T, Bastian BC (2016) CNVkit: Genome-Wide Copy Number Detection and Visualization from Targeted DNA Sequencing. PLoS Comput Biol 12(4): e1004873.

### 8. GENERALIZED STATEMENTS OF THE DISCLOSURE

The following numbered statements provide a general description of the disclosure and are not intended to limit the appended claims.

Statement 1: A method of generating a calculated cancer signature for a sample from a patient which comprises: (a) obtaining, or having obtained, a sample from the patient; (b) measuring, or having measured, a plurality of copy number alterations (CNAs) over a plurality of locations on a plurality of chromosomes; and (c) analyzing the measured CNAs using a mathematical model based on mRNA expression data and molecular subtypes, wherein the mathematical model has been validated by at least two different statistical methods so as to generate the calculated cancer signature for the sample.

Statement 2: The method of Statement 1, wherein greater than 50 CNAs are measured.

Statement 3: The method of Statement 1, wherein greater than 100 CNAs are measured.

Statement 4: The method of Statement 1, wherein between about 250 and about 400 CNAs are measured.

Statement 5: The method of any of Statements 1-4, wherein the calculated cancer signature corresponds to a somatic mutation signature.

Statement 6: The method of Statement 5, wherein the mathematical model to prepare the somatic mutation signature is based on 10 or more beta-coefficient values in Supplemental Table 6.

Statement 7: The method of any of Statements 1-4, wherein the calculated cancer signature corresponds to an mRNA expression signature.

Statement 8: The method of Statement 7, wherein the calculated cancer signature is a signature of a breast cancer subtype.

Statement 9: The method of Statement 7, wherein the mathematical model to prepare the breast cancer subtype signature is based on 10 or more beta-coefficient values in Supplemental Table 4.

Statement 10: The method of any of Statements 1-4, wherein the calculated cancer signature corresponds to a protein expression signature.

Statement 11: The method of Statement 10, wherein the mathematical model to prepare the protein expression signature is based on 10 or more beta-coefficient values in Supplemental Table 5.

Statement 12: The method of Statement 10, wherein the protein expression signature is an immunohistochemistry (IHC) signature.

Statement 13: The method of Statement 12, wherein the IHC signature is an estrogen receptor (ER), an epidermal growth factor receptor (EGFR), a human epidermal growth factor receptor 2 (HER2), a progesterone receptor (PR), or a retinoblastoma (RB) signature.

Statement 14: The method of any of Statements 1-4, wherein the calculated cancer signature corresponds to a FoundationOne® CDX result, an MAMMAPRINT® 70-GENE recurrence score, an OncotypeDX™ recurrence score, or a Prosigna® risk of recurrence score.

Statement 15: The method of Statement 14, wherein the calculated cancer signature is a FoundationOne® result and the mathematical model to prepare the FoundationOne® result is based on 10 or more beta-coefficient values in Supplemental Table 9.

Statement 16: The method of Any of Statements 1-4, wherein the calculated cancer signature is a bladder urothelial carcinoma (BLCA), cervical squamous cell carcinoma and endocervical adenocarcinoma (CESC), colon adenocarcinoma (COAD), esophageal carcinoma (ESCA), glioblastoma multiforme (GBM), head and neck squamous cell carcinoma (HNSC), kidney renal clear cell carcinoma (KIRC), kidney renal papillary cell carcinoma (KIRP), acute myeloid leukemia (LAML), brain lower grade glioma (LGG), liver hepatocellular carcinoma (LIHC), lung adenocarcinoma (LUAD), lung squamous cell carcinoma (LUSC), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), stomach adenocarcinoma (STAD), testicular germ cell tumors (TGCT), thyroid carcinoma (THCA), thymoma (THYM), or uterine corpus endometrial carcinoma (UCEC) signature.

Statement 17: The method of Statement 16, wherein the mathematical model to prepare the calculated signature is based on beta-coefficient values in Supplemental Table 14.

Statement 18: The method of any of Statements 1-17, wherein the plurality of copy number alterations (CNAs) are obtained from whole genome sequencing (WGS), whole exome sequencing (WES), or a combination thereof.

Statement 19: A method for treating a cancer patient with chemotherapy comprising the steps of: determining whether the patient has a specific cancer subtype by: (a) obtaining or having obtained a biological sample from the patient; (b) performing or having performed a gene level copy number alteration (CNA) assay on the biological sample wherein copy numbers are measured over a plurality of locations on a plurality of chromosomes; (c) comparing to results of the CNA assay to a set of standards to determine if the patient has a specific cancer subtype; and (d) if the patient has a specific cancer subtype, then administering a suitable chemotherapy regimen to the cancer patient in based on the determined cancer subtype.

Statement 20: The method of Statement 19, wherein the chemotherapy regimen is an ongoing therapeutic intervention.

Statement 21: The method of Statement 20, wherein the ongoing therapeutic intervention comprises discontinuing a specific treatment.

Statement 22: The method of any of Statements 19-21, wherein the copy number alteration (CNA) assay is obtained from whole genome sequencing (WGS), whole exome sequencing (WES), or a combination thereof.

Statement 23: A method for generating a calculated cancer signature for a cancer phenotype, the method comprising: (a) receiving a plurality of gene expression signatures and subtype information for the cancer phenotype; (b) receiving a plurality of copy number alteration (CNA) data sets for the cancer phenotype; (c) analyzing the plurality of CNA data sets with an artificial intelligence algorithm to obtain a preliminary set of CNA segment level signatures for the cancer phenotype; (d) using a gene expression training set to revise the preliminary set CNA segment level signatures and obtain a final set CNA segment level signatures; and (e) using the final set CNA segment level signatures to prepare the calculated cancer signature for the cancer phenotype.

Statement 24: The method of Statement 23, wherein the cancer phenotype is associated with a somatic mutation.

Statement 25: The method of Statement 23, wherein the cancer phenotype corresponds to a level of mRNA expression.

Statement 26: The method of Statement 23, wherein the cancer phenotype corresponds to a level of protein expression.

Statement 27: The method of Statement 26, wherein the level of protein expression corresponds to an immunohistochemistry (IHC) signature.

Statement 28: The method of any of Statements 23-26, wherein the plurality of copy number alteration (CNA) data sets are obtained from whole genome sequencing (WGS), whole exome sequencing (WES), or a combination thereof.

Statement 29: A method for generating a calculated cancer signature for a patient, the method comprising: (a) receiving copy number alteration (CNA) data for the patient; (b) receiving one or more CNA(s) signature(s) associated with a cancer phenotype, wherein the CNA signature is based on cancer expression analysis, cancer subtype information, and CNA gain/loss information; (c) processing the CNA data for patient with an algorithm utilizing the one or more CNA(s) signature(s) associated with the cancer phenotype so as to characterize the properties of the CNA data for the patient properties relative to the one or more CNA(s) signature(s); and (d) preparing a calculated cancer signature for the patient.

Statement 30: The method of Statement 29, wherein the cancer phenotype is associated with a somatic mutation.

Statement 31: The method of Statement 29, wherein the cancer phenotype corresponds to a level of mRNA expression.

Statement 32: The method of Statement 29, wherein the cancer phenotype corresponds to a level of protein expression.

Statement 33: The method of Statement 29, wherein the level of protein expression corresponds to an immunohistochemistry (IHC) signature.

Statement 34: The method of Statement 29, wherein the cancer phenotype is associated with an adrenal gland, a bladder, a bone, a breast, a cervix, a colon, a liver, a lung, a lymph, an ovarian, a pancreas, a penis, a prostate, a rectal, a salivary gland, a skin, a spleen, a testicular, a thymus gland, a thyroid, a trachea, or a uterine cancer.

Statement 35: The method of Statement 41, wherein the cancer phenotype is associated with a breast cancer.

Statement 36: The method of any of Statements 29-35, wherein the copy number alteration (CNA) data are obtained from whole genome sequencing (WGS), whole exome sequencing (WES), or a combination thereof.

Statement 37: A method for treating a subject with cancer, comprising: (a) generating a calculated cancer signature for a patient comprising: (i) receiving copy number alteration (CNA) data for the patient; (ii) receiving one or more CNA(s) signature(s) associated with a cancer phenotype, wherein the CNA signature is based on cancer expression analysis, cancer subtype information, and CNA gain/loss information; (iii) processing the CNA data for the patient with an algorithm utilizing the one or more CNA(s) signature(s) associated with the cancer phenotype so as to characterize the properties of the CNA data for the patient properties relative to the one or more CNA(s) signature(s); (iv) preparing the calculated cancer signature for the patient based on the characterized properties; and (b) treating the patient based on a treatment plan based on the calculated cancer signature.

Statement 38: The method of Statement 37, wherein the treatment is an ongoing therapeutic intervention.

Statement 39: The method of Statement 37, wherein the ongoing therapeutic intervention comprises discontinuing a specific treatment.

Statement 40: The method of any of Statements 37-39, wherein the copy number alteration (CNA) data are obtained from whole genome sequencing (WGS), whole exome sequencing (WES), or a combination thereof.

Statement 41: A device comprising a processor configured to process the patient CNA data and the one or more CNA(s) signature(s) associated with the cancer phenotype with the algorithm to generate the calculated cancer signature for the patient of Statement 29.

Statement 42: A system comprising the device of Statement 41.

Statement 43: The device of Statement 41, comprising software that comprises an algorithm to compare the patient CNA data with the one or more CNA(s) signature(s) associated with the cancer phenotype.

Statement 36: The device of any of Statements 41-43, wherein the copy number alteration (CNA) data are obtained from whole genome sequencing (WGS), whole exome sequencing (WES), or a combination thereof.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

9. COMPUTER PROGRAM

DNA-based-predictors-of-non-genetic-cancer-phenotypes

Analysis scripts and relative data used in the paper “Genetic determinants of the molecular portraits of epithelial cancers” by Xia et al.

Data

contains many .rda files ready to use in the analysis. May be found at http://github.com/xyouli/DNA-based-predictors-of-non-genetic-cancer-phenotypes

Rscripts
helper.R: This script has many R functions used in the analysis.
signature_score_and_segment_score_calculation.R: This script is used to calculate signature scores from RNA expression data and segment scores from gene-level copy number data.
association_test.R: This script is used to perform genome wide association tests between gene signatures and DNA copy number alterations.
Elastic_Net_modeling.R: This script is used to perform Elastic Net modeling analysis. All data used to build Elastic Net models are included in Data folder.

Rscript/Elastic_Net_modeling.R
57 lines (43 sloc) 1.81
library(caret)
library(glmnet)
library(ROCR)
library(doMC)
registerDoMC(cores = 8)
set.seed(1992)
source(“~/script/helper.R”)
# load data, BRCA_signatue for example
# predictor: CN_score
# response: signature_score/rppa/mutation/clinical variables
# for balanced stratification: balancing_variables
load(‘~/data/BRCA_signature_elastic_net_data.rda’)
# choose a phenotype to model
# for example, RB-LOH signature
pheno <− “UNC_RB_LOH_Median_Breast.Cancer.Res.2008_PMID.18782450”
score <− unlist(signature_score[pheno,])
score_bi <− ifelse(score >= quantile(score,0.67),1,0)
# split sample into 70% training set and 30% test set
strata <− score_bi
pik <− rep(7/10,times=length(strata))
balanced_split <−
balancedstratification(balancing_variables,strata,pik,comment = F)
train <− which(balanced_split==1)
test <− which(balanced_split==0)
trainX <− CN_score[train,]
testX <− CN_score[test,]
trainY <− score_bi[train]
testY <− score_bi[test]
glmnet_obj <− caret_wrap(trainX,trainY,testX,testY,bi = T)
# look at model performance: receiving operating curve and precision recall
curve
pred_train <− predict(glmnet_obj,newdata = trainX,type = ‘prob’)
pred_test <− predict(glmnet_obj,newdata = testX,type = ‘prob’)
pred_train <− prediction(pred_train$pos,labels = trainY)
pred_test <− prediction(pred_test$pos,labels = testY)
perf_train <− performance(pred_train,measure = ‘tpr’,x.measure = ‘fpr’)
perf_test <− performance(pred_test,measure = ‘tpr’,x.measure = ‘fpr’)
auc_train <− signif(performance(pred_train,measure =
‘auc’)@y.values[[1]][1],2)
auc_test <− signif(performance(pred_test,measure = ‘auc’)@y.values[[1]][1],2)
plot_ROC(perf_train,perf_test,auc_train,auc_test,pheno)
# look at feature landscape
beta <− as.matrix(coef(glmnet_obj$finalModel,glmnet_obj$bestTune$lambda))
beta <− beta[−1,1]
plot_seg_ss(beta,pheno)
DNA-based-predictors-of-non-genetic-cancer-phenotypes/Rscript/association_test.R
27 lines (22 sloc) 1.14 KB
# Test for associations between gene signatures and DNA CNA
# Given a signature_score matrix and
# gene-level CNA matrix CN_score: continuous CNA score for each gene,
# CN_gain: binary matrix where 1 is copy number gain for a gene and 0 is no
gain
# CN_loss: binary matrix where 1 is copy number loss for a gene and 0 is no
loss
# Fisher's exact test and spearman correlation test/linear model control for
subtypes
# choose a gene signature to test, for example RB-LOH signature
load(‘BRCA_association_test_data.rda’)
pheno <− “UNC_RB_LOH_Median_Breast.Cancer.Res.2008_PMID.18782450”
score <− unlist(signature_score [pheno,])
p_value <− sigCNTest(score, CN_score, CN_gain, CN_loss)
# Benjamini-Hochberg correct p values
p_value_adj <− apply(p_value,2,function(x){return(p.adjust(x,method = “BH”))})
# log10 transform adjusted p values
log_p <− −log(p_value_adj,10)
# plot association landscape
v <− vertical_lines
# for unadjusted associations
p <− log_p[,1:4]
# for subtype-adjusted associations
p <− log_p[,c(5:6,3:4)]
P_Plot(p,main = ‘UNC_RB_LOH_Median_Breast.Cancer.Res.2008_PMID.18782450’,y1 =
35,y2=50,label_up = seq(0,35,by=10),label_down = seq(10,50,by=10))
DNA-based-predictors-of-non-genetic-cancer-
phenotypes/Rscript/signature_score_and_segment_score_calculation.R
48 lines (34 sloc) 2.2 KB
# This script is for calculating gene signature scores based on RNA and
segment scores based on gene-level DNA CNA
# Given a gene expression data matrix (gene X sample): edata
# rows are genes in Entrez ID and columns are samples
# run calc_signatures
signature_score <−
calc_signatures(edata,“~/data/gene_signatures_20170111.gmt”,method = “median”)
# find NA signatures
NAsig <− rownames(signature_score[is.na(signature_score[,1]),])
# remove NA signatures
i <− match(NAsig,rownames(signature_score))
signature_score <− signature_score[−i,]
# CD103_Ratio
CD103_pos <−
signature_score[rownames(signature_score)==“CD103_Positive_Median_Cancer.Cell.
2014_PMID.25446897”]
CD103_neg <−
signature_score[rownames(signature_score)==“CD103_Negative_Median_Cancer.Cell.
2014_PMID.25446897”]
CD103_ratio <− CD103_pos − CD103_neg # log2 scale division
signature_score <− rbind(signature_score,CD103_ratio)
rownames(signature_score)[nrow(signature_score)] <−
“CD103_Ratio_Cancer.Cell.2014_PMID.25446897”
# differentiation score
diff_centroid <−
read.table(“~/data/special_gene_signature_training_sets/UNC_Differentiation.Sc
ore_Model_BCR.2010_PMID.20813035”,sep = ‘\t’,header = T,row.names =
1,check.names = F)
diff_score <− assignDiffScore.dwd(diff_centroid,edata)
signature_score <− rbind(signature_score,diff_score)
rownames(signature_score)[nrow(signature_score)] <−
“UNC_Differentiation.Score_Model_BCR.2010_PMID.20813035”
# oncotype DX score
oncotype <− GHI_RS(edata)
signature_score <− rbind(signature_score,diff_score)
rownames(signature_score)[nrow(signature_score)] <−
“GHI_RS_Model_NJEM.2004_PMID.15591335”
save(signature_score,file = ‘signature_score.rda’)
# For special signatures calculated as correlation to predetermined gene
centroids
# All traing sets files are included in the
~/data/special_gene_signature_training_sets folder
# For calculation of such special signature, DWD was used to merge current
edata with
# training set, then DWD prediction tool was used to compute pearson/spearman
correlation/euclidean distance
# for each sample
# Given a gene-level CNA score matrix (gene X sample): CNdata
# rows are genes in Entrez ID and columns are samples
segment_score <− calc_segments(CNdata,‘CNA_segments.gmt’,method = ‘mean’)

1. A method of generating a calculated cancer signature for a sample from a patient which comprises:
(a) obtaining, or having obtained, a sample from the patient;
(b) measuring, or having measured, a plurality of copy number alterations (CNAs) over a plurality of locations on a plurality of chromosomes; and
(c) analyzing the measured CNAs using a mathematical model based on mRNA expression data and molecular subtypes, wherein the mathematical model has been validated by at least two different statistical methods so as to generate the calculated cancer signature for the sample.

2. The method of claim 1, wherein greater than 50 CNAs are measured.

3. The method of claim 1, wherein greater than 100 CNAs are measured.

4. The method of claim 1, wherein between about 250 and about 400 CNAs are measured.

5. The method of claim 1, wherein the calculated cancer signature corresponds to a somatic mutation signature.

6. (canceled)

7. The method of claim 1, wherein the calculated cancer signature corresponds to an mRNA expression signature.

8. The method of claim 1, wherein the calculated cancer signature is a signature of a breast cancer subtype.

9. (canceled)

10. The method of claim 1, wherein the calculated cancer signature corresponds to a protein expression signature.

11. (canceled)

12. The method of claim 10, wherein the protein expression signature is an immunohistochemistry (IHC) signature.

13. The method of claim 12, wherein the IHC signature is an estrogen receptor (ER), an epidermal growth factor receptor (EGFR), a human epidermal growth factor receptor 2 (HER2), a progesterone receptor (PR), or a retinoblastoma (RB) signature.

14. The method of claim 1, wherein the calculated cancer signature corresponds to a FoundationOne® CDX result, an MAMMAPRINT® 70-GENE recurrence score, an OncotypeDX™ recurrence score, or a Prosigna® risk of recurrence score.

15. (canceled)

16. The method of claim 1, wherein the calculated cancer signature is a bladder urothelial carcinoma (BLCA), cervical squamous cell carcinoma and endocervical adenocarcinoma (CESC), colon adenocarcinoma (COAD), esophageal carcinoma (ESCA), glioblastoma multiforme (GBM), head and neck squamous cell carcinoma (HNSC), kidney renal clear cell carcinoma (KIRC), kidney renal papillary cell carcinoma (KIRP), acute myeloid leukemia (LAML), brain lower grade glioma (LGG), liver hepatocellular carcinoma (LIHC), lung adenocarcinoma (LUAD), lung squamous cell carcinoma (LUSC), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), stomach adenocarcinoma (STAD), testicular germ cell tumors (TGCT), thyroid carcinoma (THCA), thymoma (THYM), or uterine corpus endometrial carcinoma (UCEC) signature.

17. (canceled)

18. The method of claim 1, wherein the plurality of copy number alterations (CNAs) are obtained from whole genome sequencing (WGS), whole exome sequencing (WES), or a combination thereof.

19.-28. (canceled)

29. A method for generating a calculated cancer signature for a patient, the method comprising:
(a) receiving copy number alteration (CNA) data for the patient;
(b) receiving one or more CNA(s) signature(s) associated with a cancer phenotype, wherein the CNA signature is based on cancer expression analysis, cancer subtype information, and CNA gain/loss information;
(c) processing the CNA data for patient with an algorithm utilizing the one or more CNA(s) signature(s) associated with the cancer phenotype so as to characterize the properties of the CNA data for the patient properties relative to the one or more CNA(s) signature(s); and
(d) preparing a calculated cancer signature for the patient.

30. (canceled)

31. (canceled)

32. (canceled)

33. (canceled)

34. The method of claim 29, wherein the cancer phenotype is associated with an adrenal gland, a bladder, a bone, a breast, a cervix, a colon, a liver, a lung, a lymph, an ovarian, a pancreas, a penis, a prostate, a rectal, a salivary gland, a skin, a spleen, a testicular, a thymus gland, a thyroid, a trachea, or a uterine cancer.

35. (canceled)

36. The method of claim 29, wherein the copy number alteration (CNA) data are obtained from whole genome sequencing (WGS), whole exome sequencing (WES), or a combination thereof.

37. A method for treating a subject with cancer, comprising:
(a) generating a calculated cancer signature for a patient comprising:
(b) receiving copy number alteration (CNA) data for the patient;
(c) receiving one or more CNA(s) signature(s) associated with a cancer phenotype, wherein the CNA signature is based on cancer expression analysis, cancer subtype information, and CNA gain/loss information;
(d) processing the CNA data for the patient with an algorithm utilizing the one or more CNA(s) signature(s) associated with the cancer phenotype so as to characterize the properties of the CNA data for the patient properties relative to the one or more CNA(s) signature(s);
(e) preparing the calculated cancer signature for the patient based on the characterized properties; and
(f) treating the patient based on a treatment plan based on the calculated cancer signature.

38. (canceled)

39. (canceled)

40. (canceled)

41. A device comprising a processor configured to process the patient CNA data and the one or more CNA(s) signature(s) associated with the cancer phenotype with the algorithm to generate the calculated cancer signature for the patient of claim 29.

42. A system comprising the device of claim 41.

43. The device of claim 41, comprising software that comprises an algorithm to compare the patient CNA data with the one or more CNA(s) signature(s) associated with the cancer phenotype.
